(19) United States
(12) Patent Application Publication
(10) Pub. No.: US 2022/0372097 A1
(43) Pub. Date: Nov. 24, 2022

(54) IL-15 FUSION PEPTIDES USED TO TREAT CANCER

(71) Applicants: PROSTATE CANCER RESEARCH CENTRE, LONDON (GB); KING'S COLLEGE LONDON, LONDON (GB)

(72) Inventors: CHRISTINE GALUSTIAN, LONDON (GB); RICHARD SMITH, LONDON (GB); DOROTA SMOLAREK, LONDON (GB); EFTHYMIA PAPAEVANGELOU, LONDON (GB); PROKAR DASGUPTA, LONDON (GB)

(21) Appl. No.: 17/762,358

(22) PCT Filed: Sep. 25, 2020
(86) PCT No.: PCT/GB2020/052328

§ 371 Date: Mar. 21, 2022

(30) Foreign Application Priority Data
Sep. 25, 2019 (GB) 1913804.9

(51) Int. Cl. C07K 14/54; A61P 35/00

(52) U.S. Cl. CPC C07K 14/5443; A61P 35/00; C07K 2319/01; A61K 38/00

(57) ABSTRACT

The present invention is directed to a fusion polypeptide, the polypeptide comprising: a. an interleukin-15 (IL-15); and b. an IL-15 activity-promoting sequence, wherein said sequence: is between 10 and 60 amino acid residues in length; and increases CD8+ T-cell proliferation 5 by the IL-15. Also provided are nucleic acids encoding the fusion polypeptide, associated methods of producing the fusion polypeptide, pharmaceutical compositions and kits comprising the same, and therapeutic uses thereof.

The present invention relates to polypeptide therapeutics, such as polypeptide therapeutics for treating cancer.

Cancer is a serious ongoing public health concern accounting for 7.6 million of the 58 million deaths worldwide in 2005. Cancer incidence has since increased each year, with a prediction that it will account for 11.4 million deaths in 2030.

Solid tumours account for the majority of the aforementioned cancers. Solid tumours originate from an abnormal mass of tissue that does not contain cysts or liquid areas. Such tumours can be benign (non-cancerous), however in the context of solid tumour cancer, the solid tumours are malignant (cancerous). Solid tumours can be classified into three groups based on the type of cell from which they are composed: sarcomas; carcinomas; and lymphomas. Lymphomas develop in the glands or nodes of the lymphatic system and are distinguished from leukaemias, which are referred to as liquid cancers. Sarcomas are cancers that originate in the supportive and connective tissue, e.g. bones, tendons, cartilage, muscle, and fat. Carcinoma refers to a malignant neoplasm of epithelial origin or cancer of the internal or external lining of the body. In other words, carcinomas are malignancies of epithelial tissue. Carcinomas account for 80-90% of all cancer cases.

One such carcinoma is prostate cancer. Cancer of the prostate is the most common cancer in men, with age being a key risk factor as ˜99% of cases occur in males over 50. Early-stage prostate cancer is typically asymptomatic, but urinary dysfunction symptoms, such as frequent/difficult/painful urination, heamturia, and nocturia, may be present. As prostate cancer progresses symptoms may include sexual dysfunction. Late-stage prostate cancer is associated with cancer cell metastasis, commonly leading to secondary tumours in the bones and lymph nodes. Symptoms may include bone pain, tingling, leg weakness, and urinary and faecal incontinence. Prostate cancer is frequently detected at an early localised stage through a variety of screening procedures, including detection of prostate-specific antigen (PSA), prostate imaging, digital rectal examination, and biopsy. Surgical removal following, or prior to, chemotherapy, hormonal therapies and radiotherapy can be effective and has become routine clinical practice. However, side effects may remain, including immunosuppression, neutropenia, and thrombocytosis. Moreover, genitourinary damage can occur in over 50% of prostate cancer patients undergoing prostectomy. Prostate cancer can be particularly difficult to treat and, in particular, the prostate cancer microenvironment is immunosuppressive, thus reducing the effectiveness of the immune system at targeting and destroying prostate cancer cells. Thus, there is a need for an improved therapeutic to treat cancer generally, and prostate cancer in particular.

TH1 cytokines, including Interleukin-2 (IL-2) and Interleukin-15 (IL-15) have been employed in the treatment of cancers.

IL-15 is a member of the four-a-helix bundle family of cytokines and plays a role in both innate and adaptive immunity mediated by binding to a cell-surface receptor. The receptor comprises three subunits: IL-15 receptor (IL-15R) α, IL-2Rβ (also known as IL-15Rβ, CD122, and p75), and γ_{c} (also known as CD132 and p65). IL-15 has been shown to function in trans where the receptor is formed from an IL-15Rα subunit of a first cell and a IL-2Rβ and γ_{c} subunit of a second cell, or in cis where the receptor is formed from an IL-15Rα subunit, IL-2Rβ subunit, and γ_{c} subunit on the same cell.

IL-15 has been shown to be a particularly effective therapeutic, but is associated with a number of disadvantages including systemic toxicity. Thus, there is a need for an IL-15 therapeutic with improved efficacy, thereby allowing for the administration of lower dosages and reduced systemic toxicity.

The present invention overcomes one or more of the above-mentioned problems.

The present inventors have surprisingly found that fusing interleukin-15 to an IL-15 activity-promoting peptide improves the activity of IL-15. Without wishing to be bound by theory, it is believed that an IL-15 activity-promoting peptide of the invention stabilises the interaction between IL-15 and its receptor, optionally providing for more freedom of movement for the IL-15 molecule when interacting with its receptor (either in the cis or trans configuration). Advantageously, this allows for the administration of lower doses of the fusion polypeptide of the invention in the treatment of cancer, thereby reducing side-effects associated with wild-type IL-15, such as systemic toxicity.

In one aspect the invention provides a fusion polypeptide (e.g. for treating cancer), the polypeptide comprising:
a. an interleukin-15 (IL-15); and
b. an IL-15 activity-promoting sequence, wherein said sequence:
   is between 10 and 60 amino acid residues in length; and
   increases CD8+ T-cell proliferation by the IL-15.

A fusion polypeptide of the present invention comprises interleukin-15. Preferably, the IL-15 is mature IL-15, which lacks the signal peptide (e.g. amino acids 1-29) and propeptide (e.g. amino acids 30-48) of an IL-15 precursor. A reference human IL-15 precursor is shown herein as SEQ ID NO: 1.

An IL-15 herein may be a mammalian IL-15 or a functional fragment thereof, e.g. a human IL-15 or functional fragment thereof, a primate IL-15 or a functional fragment thereof, or a murine IL-15 or a functional fragment thereof. An IL-15 is preferably a human IL-15 or a functional fragment thereof. In one embodiment, an IL-15 comprises a polypeptide sequence having at least 70% sequence identity to SEQ ID NO: 2 or 3. Preferably, an IL-15 comprises a polypeptide sequence having at least 80% or 90% sequence identity to SEQ ID NO: 2 or 3. More preferably, an IL-15 comprises a polypeptide sequence having at least 95% sequence identity to SEQ ID NO: 2 or 3. In a particularly preferred embodiment an IL-15 comprises (more preferably consists of) SEQ ID NO: 2 or 3, more preferably an IL-15 comprises (more preferably consists of) SEQ ID NO: 3.

An IL-15 may comprise (or consist of) a polypeptide sequence having at least 70% sequence identity to any one of SEQ ID NOs: 25-27. In one embodiment an IL-15 of the invention comprises (or consists of) a polypeptide sequence having at least 80% or 90% sequence identity to any one of SEQ ID NOs: 25-27. Preferably, an IL-15 of the invention comprises (or consists of) a polypeptide sequence having at least 95% sequence identity to any one of SEQ ID NOs: 25-27. More preferably, an IL-15 of the invention comprises (more preferably consists of) any one of SEQ ID NOs: 25-27.

A functional fragment of IL-15 is a truncation of IL-15 having IL-15 activity. In one embodiment a functional fragment of IL-15 has the ability to promote CD8+ T-cell proliferation and/or differentiation. In one embodiment, a functional fragment of IL-15 has the ability to promote natural killer (NK) cell proliferation and/or differentiation. In one embodiment, a functional fragment of IL-15 has the ability to promote B-cell proliferation and/or differentiation. Preferably, the functional fragment of II-15 has the ability to promote CD8+ T-cell proliferation and/or differentiation, natural killer (NK) cell proliferation and/or differentiation, and/or B-cell proliferation and/or differentiation.

An IL-15 activity-promoting sequence is between 10 and 60 amino acid residues in length. The IL-15 activity-promoting sequence is preferably a peptide sequence.

For the avoidance of any doubt, where a range is mentioned, said range encompasses the numbers that form the end point thereof. For example, a sequence that is between 10 and 60 amino acid residues in length encompasses a sequence that is 10 amino acid residues in length as well as a sequence that is 60 amino acid residues in length.

An IL-15 activity-promoting sequence may be at least 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59 amino acids in length. An IL-15 activity-promoting sequence may be less than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12 or 11 amino acids in length.

Preferably, an IL-15 activity-promoting sequence is at least 32 amino acid residues in length.

In one embodiment an IL-15 activity-promoting sequence is at least 15, 20, 25 or 30 amino acid residues in length and up to 60, 55, or 50 amino acid residues in length. In one embodiment an IL-15 activity-promoting sequence is between 25-55 amino acid residues in length. Preferably, an IL-15 activity-promoting sequence is between 40-50 amino acid residues in length. More preferably, an IL-15 activity-promoting sequence is 45-50 amino acid residues amino acid residues in length, e.g. 46 amino acid residues in length.

An IL-15 activity-promoting sequence may comprise at least one cysteine or lysine residue. Preferably, an IL-15 activity-promoting sequence comprises at least one cysteine residue, more preferably one cysteine residue. The at least one cysteine or lysine residue may be located at or near to (preferably at) the N- or C-terminus of the activity-promoting sequence (when referring to the primary polypeptide sequence of the IL-15 activity-promoting sequence). The location of the at least one cysteine or lysine residue may suitably be determined based on the position of the IL-15 activity-promoting sequence respective to IL-15. In other words, where the IL-15 activity-promoting sequence is located C-terminal to IL-15 (when referring to the primary polypeptide sequence of the fusion polypeptide), the at least one cysteine or lysine residue may be located at or near to (preferably at) the C-terminus of the activity-promoting sequence, while where the IL-15 activity-promoting sequence is located N-terminal with respect to IL-15 (when referring to the primary polypeptide sequence of the fusion polypeptide), the at least one cysteine or lysine residue may be located at or near to (preferably at) the N-terminus of the activity-promoting sequence. Preferably, the at least one cysteine or lysine residue is located at or near to (preferably at) the C-terminus of the IL-15 activity-promoting sequence.

An IL-15 activity-promoting sequence of the invention promotes at least a CD8+ T-cell proliferation activity of IL-15. In other words, the IL-15 activity-promoting sequence increases CD8+ T-cell proliferation by the IL-15 when compared to an equivalent polypeptide comprising IL-15 (preferably consisting of an identical IL-15 polypeptide) and lacking the IL-15 activity-promoting sequence.

The term “increases CD8+ T-cell proliferation by the IL-15” as used herein refers to an increase in CD8+ T-cell proliferation as measured in vitro using the “CTLL-2 assay” described herein. Preferably, the increase is a statistically-significant increase in CD8+ T-cell proliferation as measured in vitro using the “CTLL-2 assay” described herein.

Statistical-significance herein may be determined using any suitable technique, preferably 1-way ANOVA or the post-hoc Newman-Keuls method.

The “CTLL-2 assay” is carried out by:

a) culturing murine CTLL-2 cells at a concentration of 5×10⁵ cells/ml in 96 well plates (5×10⁴ cells per well in a volume of 100 ul) for 72 hours in the presence of an IL-15 polypeptide fused to a test peptide (IL-15-test peptide fusion) at 37° C.;

b) incubating the cells with MTS (5-[3-(carboxymethoxy)phenyl]-3-(4,5-dimethyl-2-thiazolyl)-2-(4-sulfophenyl)-2H-tetrazolium inner salt) for 3-4 hours (at the 72 hour time point);

c) quantifying the number of cells by colorimetry at an absorbance of 490 nm;

d) comparing the number of CTLL-2 cells quantified in step c) with the number of CTLL-2 cells in a control sample that has been assayed under the same conditions but in the presence of wild-type IL-15 (e.g. SEQ ID NO: 2 or 3); and

e) wherein the test peptide increases CD8+ T-cell proliferation by the IL-15 when the number of CTLL-2 cells quantified in step c) is greater than (preferably statistically-significantly greater than) the number of CTLL-2 cells quantified in the control sample; or wherein the test peptide does not increase or decreases CD8+ T-cell proliferation by the IL-15 when the number of CTLL-2 cells quantified in step c) is substantially the same (e.g. where there is no statistically-significant difference, preferably no difference) or less than (preferably is statistically-significantly less than) the number of CTLL-2 cells quantified in the control sample.

In one embodiment a test peptide increases CD8+ T-cell proliferation by the IL-15 when at a concentration of 0.1 ng/ml-1 ng/ml (preferably 0.2-0.5 ng/ml, more preferably at 0.2-0.4 ng/ml) of the IL-15-test peptide fusion, the number of CTLL-2 cells quantified in step c) is greater than the number of CTLL-2 cells quantified in the control sample (wherein the wild-type IL-15 of the control sample has been used at the same concentration); or wherein the test peptide does not increase or decreases CD8+ T-cell proliferation by the IL-15 when at a concentration of 0.1 ng/ml-1 ng/ml (preferably 0.2-0.5 ng/ml, more preferably at 0.2-0.4 ng/ml) of the IL-15-test peptide fusion, the number of CTLL-2 cells quantified in step c) is substantially the same or less than the number of CTLL-2 cells quantified in the control sample (wherein the wild-type IL-15 of the control sample has been used at the same concentration).

Where a test peptide does increase (preferably statistically-significantly increases) CD8+ T-cell proliferation by the IL-15 as determined by the “CTLL-2 assay”, said test peptide is an IL-15 activity-promoting sequence in accordance with the invention.

Where a test peptide does not increase or decreases CD8+ T-cell proliferation by the IL-15 as determined by the “CTLL-2 assay”, said test peptide is not an IL-15 activity-promoting sequence in accordance with the invention. Preferably, where a test peptide does not statistically-significantly increase or decreases (preferably statistically significantly decreases) CD8+ T-cell proliferation by the IL-15 as determined by the “CTLL-2 assay”, said test peptide is not an IL-15 activity-promoting sequence in accordance with the invention.

In one embodiment an increase in CD8+ T-cell proliferation by the IL-15 is an increase of at least 1%, 2%, 5%, 10%, 15%, 20%, 25%, 30%, 40%, 50%, 60%, 70%, 80%, 90%, 100% or 120% when compared to an equivalent polypeptide comprising IL-15 (preferably consisting of an identical IL-15 polypeptide) and lacking the IL-15 activity-promoting sequence.

CTLL-2 cells are commercially available from LGC Standards, UK (ATCC® TIB-214™). Likewise, MTS reagent is commercially available from Promega (CellTiter 96® AQueous One Solution Cell Proliferation Assay).

The skilled person will appreciate that the CTLL-2 assay may be modified such that the control used in step d) is a positive control, e.g. a fusion polypeptide exemplified herein, such as SEQ ID NO: 5. In such cases, when the number of CTLL-2 cells quantified in step c) is substantially the same (e.g. where there is no statistically-significant difference, preferably no difference) or greater than (preferably is statistically-significantly greater than) the number of CTLL-2 cells quantified in the control sample, the test peptide is determined to increase CD8+ T-cell proliferation by the IL-15. Similarly, when the number of CTLL-2 cells quantified in step c) is less than (preferably statistically-significantly less than) the number of CTLL-2 cells quantified in the control sample, the test peptide is determined to not increase CD8+ T-cell proliferation by the IL-15.

Preferably, an IL-15 activity-promoting sequence of the invention does not increase receptor-independent binding of the polypeptide to a cell surface when compared to an equivalent polypeptide comprising IL-15 (preferably consisting of an identical IL-15 polypeptide) lacking an IL-15 activity-promoting sequence.

The term “does not increase receptor-independent binding of the polypeptide to a cell surface” means that an IL-15 activity-promoting sequence does not substantially increase receptor-independent binding of the polypeptide to a cell surface as determined using the “cell surface binding assay” described herein. The receptor may be any receptor to which wild-type IL-15 binds, such as IL15Rα, IL2Rβ, γC or combinations thereof.

In one embodiment an increase in receptor-independent binding of the polypeptide to a cell surface herein means a statistically-significant increase in receptor-independent binding to a cell surface as determined using the “cell surface binding assay” described herein.

The “cell surface binding assay” is carried out by:

a) incubating 8×10⁶ Jurkat or sheep red blood cells with 2 ug of an IL-15 polypeptide fused to a test peptide (IL-15-test peptide fusion) at 25° C. for 20 minutes;

b) washing the cells with PBS (phosphate buffered saline) containing 2% FCS (foetal calf serum);

c) centrifuging at 1800 rpm for 5 minutes at room 25° C. and removing any supernatant;

e) incubating the cells with 2 ul of mouse anti-human IL-15 PE-conjugated antibody in darkness for 20 minutes at 4° C.;

f) washing the cells with PBS containing 2% FCS;

g) centrifuging at 1800 rpm for 5 minutes at room 4° C. and removing any supernatant;

h) washing the cells with PBS containing 2% FCS;

i) centrifuging at 1800 rpm for 5 minutes at room 4° C. and removing any supernatant;

j) resuspending the cells in 400 μl PBS containing 2% FCS;

k) quantifying binding of the IL-15-test peptide fusion to the cells by flow cytometry;

I) comparing the quantified binding of k) with the quantified binding in a control sample that has been assayed under the same conditions but in the absence of the IL-15-test peptide fusion or in the presence of wild-type IL-15 (e.g. SEQ ID NO: 2 or 3) (preferably in the absence of the IL-15-test peptide fusion); and

m) wherein the test peptide does not increase receptor-independent binding of the polypeptide to a cell surface when the quantified binding is substantially the same (e.g. where there is no statistically-significant difference, preferably where the quantified binding is identical) or less (preferably statistically-significantly less) when compared to the quantified binding of the control sample; or wherein the test peptide increases receptor-independent binding of the polypeptide to a cell surface when the quantified binding is greater (preferably statistically-significantly greater) when compared to the quantified binding of the control sample.

Where a test peptide does not increase (e.g. does not statistically-significantly increase) or decreases receptor-independent binding of the polypeptide to a cell surface as determined by the “cell surface binding assay”, said test peptide may be selected as an IL-15 activity-promoting sequence in accordance with the invention.

Where a test peptide increases (e.g. statistically-significantly increases) receptor-independent binding of the polypeptide to a cell surface as determined by the “cell surface binding assay”, said test peptide may be rejected as not being an IL-15 activity-promoting sequence in accordance with the invention.

A PE conjugated antibody for use in the assay can be obtained from R&D Systems (Cat. Number IC2471P).

Sheep red blood cells for use in the assay can be obtained from Antibodies-Online (Cat. Number ABIN770405). Jurkat cells for use in the assay can be obtained from LGC Standards, UK (ATCC® TIB-152™).

The skilled person will appreciate that the cell surface binding assay may be modified such that the control used in step I) is a positive control, e.g. a fusion polypeptide exemplified herein, such as SEQ ID NO: 5. In such cases, when the quantified binding is substantially the same (e.g. where there is no statistically-significant difference, preferably where the quantified binding is identical) or less (preferably statistically-significantly less) when compared to the quantified binding of the control sample, the test peptide is determined to not increase receptor-independent binding of the polypeptide to a cell surface. Similarly, when the quantified binding is greater (preferably statistically-significantly greater) when compared to the quantified binding of the control sample, the test peptide is determined to increase receptor-independent binding of the polypeptide to a cell surface.

An IL-15 activity-promoting sequence can be positioned either C-terminal or N-terminal to the IL-15 (when referring to the primary polypeptide sequence of a fusion polypeptide of the invention). In a preferred embodiment, a fusion polypeptide comprises a N-terminal IL-15 and a C-terminal IL-15 activity-promoting sequence. Preferably, the N-terminal amino acid residue of an IL-15 activity-promoting sequence is immediately C-terminal to the C-terminal amino acid residue of an IL-15 in the primary polypeptide sequence of a fusion polypeptide of the invention.

In one embodiment an IL-15 activity-promoting sequence of the invention comprises (or consists of) a polypeptide sequence having at least 70% sequence identity to SEQ ID NO: 4. In one embodiment an IL-15 activity-promoting sequence of the invention comprises (or consists of) a polypeptide sequence having at least 80% or 90% sequence identity to SEQ ID NO: 4. Preferably, an IL-15 activity-promoting sequence of the invention comprises (or consists of) a polypeptide sequence having at least 95% sequence identity to SEQ ID NO: 4. More preferably, an IL-15 activity-promoting sequence comprises (more preferably consists of) SEQ ID NO: 4.

In one embodiment an IL-15 activity-promoting sequence of the invention comprises (or consists of) a polypeptide sequence having at least 70% sequence identity to SEQ ID NO: 9. In one embodiment an IL-15 activity-promoting sequence of the invention comprises (or consists of) a polypeptide sequence having at least 80% or 90% sequence identity to SEQ ID NO: 9. Preferably, an IL-15 activity-promoting sequence of the invention comprises (or consists of) a polypeptide sequence having at least 95% sequence identity to SEQ ID NO: 9. More preferably, an IL-15 activity-promoting sequence comprises (more preferably consists of) SEQ ID NO: 9.

While the IL-15 activity-promoting sequence of the invention may comprise (or consist of) SEQ ID NO: 4 or 9, an IL-15 activity-promoting sequence comprising (or consisting of) SEQ ID NO: 4 is preferred.

In one aspect, the invention provides a fusion polypeptide, the polypeptide comprising: an interleukin-15 (IL-15); and a peptide, wherein the peptide is between 10 and 60 amino acid residues in length and has at least 70% sequence identity to SEQ ID NO: 4 or 9 (preferably at least 70% sequence identity to SEQ ID NO: 4).

The peptide may be at least 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59 amino acids in length. The peptide may be less than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12 or 11 amino acids in length.

Preferably, the peptide is at least 32 amino acid residues in length.

In one embodiment the peptide is at least 15, 20, 25 or 30 amino acid residues in length and up to 60, 55, or 50 amino acid residues in length. In one embodiment the peptide is between 25-55 amino acid residues in length. Preferably, the peptide is between 40-50 amino acid residues in length. More preferably, the peptide is 45-50 amino acid residues amino acid residues in length, e.g. 46 amino acid residues in length.

The peptide may comprise at least one cysteine or lysine residue. Preferably, the peptide comprises at least one cysteine residue, more preferably one cysteine residue. The at least one cysteine or lysine residue may be located at or near to (preferably at) the N- or C-terminus of the peptide (when referring to the primary polypeptide sequence of the peptide). The location of the at least one cysteine or lysine residue may suitably be determined based on the position the peptide respective to IL-15. In other words, where the peptide is located C-terminal to IL-15 (when referring to the primary polypeptide sequence of the fusion polypeptide), the at least one cysteine or lysine residue may be located at or near to (preferably at) the C-terminus of the peptide, while where the peptide is located N-terminal with respect to IL-15 (when referring to the primary polypeptide sequence of the fusion polypeptide), the at least one cysteine or lysine residue may be located at or near to (preferably at) the N-terminus of the peptide. Preferably, the at least one cysteine or lysine residue is located at or near to (preferably at) the C-terminus of the peptide.

A fusion polypeptide of the present invention may comprise (or consist of) a polypeptide sequence having at least 70% sequence identity to SEQ ID NO: 5. In one embodiment a fusion polypeptide of the invention comprises (or consists of) a polypeptide sequence having at least 80% or 90% sequence identity to SEQ ID NO: 5. Preferably, a fusion polypeptide of the invention comprises (or consists of) a polypeptide sequence having at least 95% sequence identity to SEQ ID NO: 5. More preferably, a fusion polypeptide of the invention comprises (more preferably consists of) SEQ ID NO: 5.

A fusion polypeptide of the present invention may comprise (or consist of) a polypeptide sequence having at least 70% sequence identity to SEQ ID NO: 10. In one embodiment a fusion polypeptide of the invention comprises (or consists of) a polypeptide sequence having at least 80% or 90% sequence identity to SEQ ID NO: 10. Preferably, a fusion polypeptide of the invention comprises (or consists of) a polypeptide sequence having at least 95% sequence identity to SEQ ID NO: 10. More preferably, a fusion polypeptide of the invention comprises (more preferably consists of) SEQ ID NO: 10.

A fusion polypeptide of the present invention may comprise (or consist of) a polypeptide sequence having at least 70% sequence identity to SEQ ID NO: 28. In one embodiment a fusion polypeptide of the invention comprises (or consists of) a polypeptide sequence having at least 80% or 90% sequence identity to SEQ ID NO: 28. Preferably, a fusion polypeptide of the invention comprises (or consists of) a polypeptide sequence having at least 95% sequence identity to SEQ ID NO: 28. More preferably, a fusion polypeptide of the invention comprises (more preferably consists of) SEQ ID NO: 28.

While the fusion polypeptide may comprise (or consist of) SEQ ID NO: 5, 10 or 28, a fusion polypeptide comprising (or consisting of) SEQ ID NO: 5 is preferred.

An IL-15 activity-promoting sequence of the invention advantageously provides a convenient scaffold to which one or more therapeutically-relevant functional groups can be conjugated, without significantly affecting the activity of the IL-15 portion of the fusion polypeptide.

In one embodiment, a membrane binding element may be conjugated to an IL-15 activity-promoting sequence, thereby providing a fusion polypeptide that is capable of receptor-independent cell surface binding. Thus, the fusion polypeptide comprising a membrane binding element is capable of binding to a membrane of a cell, such as a cancer cell described herein. Advantageously, such a fusion polypeptide can be administered locally so that the fusion polypeptide has an effect at a specific location rather than having a systemic effect.

A membrane binding element may be any suitable molecule capable of binding to a cell membrane. Such a molecule may be identified using the “cell surface binding assay” modified as follows:

a) incubating 8×10⁶ Jurkat or sheep red blood cells with a putative membrane binding element conjugated to a fusion polypeptide of the invention (e.g. SEQ ID NO: 5) at 25° C. for 20 minutes;

b) washing the cells with PBS (phosphate buffered saline) containing 2% FCS (foetal calf serum);

c) centrifuging at 1800 rpm for 5 minutes at room 25° C. and removing any supernatant;

e) incubating the cells with 2 ul of mouse anti-human IL-15 PE-conjugated antibody in darkness for 20 minutes at 4° C.;

f) washing the cells with PBS containing 2% FCS;

g) centrifuging at 1800 rpm for 5 minutes at room 4° C. and removing any supernatant;

h) washing the cells with PBS containing 2% FCS;

i) centrifuging at 1800 rpm for 5 minutes at room 4° C. and removing any supernatant;

j) resuspending the cells in 400 μl PBS containing 2% FCS;

k) quantifying binding of the putative membrane binding element-fusion polypeptide conjugate to the cells by flow cytometry;

I) comparing the quantified binding of k) with the quantified binding in a control sample that has been assayed under the same conditions but with a fusion polypeptide in the absence of the putative membrane binding element (e.g. SEQ ID NO: 5); and

m) wherein the putative membrane binding element is confirmed as a membrane binding element when the quantified binding is greater (preferably statistically-significantly greater) when compared to the quantified binding of the control sample; or wherein the putative membrane binding element is confirmed not to be a membrane binding element when the quantified binding is substantially the same (e.g. where there is no statistically-significant difference, preferably where the quantified binding is identical) or less (preferably statistically-significantly less) when compared to the quantified binding of the control sample.

The skilled person will appreciate that the cell surface binding assay may be modified such that the control used in step I) is a positive control, e.g. a fusion polypeptide exemplified herein, such as SEQ ID NO: 7. In such cases, when the quantified binding is substantially the same (e.g. where there is no statistically-significant difference, preferably where the quantified binding is identical) or greater (preferably statistically-significantly greater) when compared to the quantified binding of the control sample, the putative membrane binding element is confirmed to be a membrane binding element. Similarly, when the quantified binding is less (preferably statistically-significantly less) when compared to the quantified binding of the control sample, the putative membrane binding element is confirmed not to be a membrane binding element.

Suitable naturally-occurring membrane binding elements are well known to those skilled in the art, either as components of proteins that mediate membrane interactions or as membrane components such as sterols or sphingolipids.

The membrane binding element should be sufficiently hydrophilic to ensure that, when conjugated to a fusion polypeptide of the invention, said polypeptide exhibits an adequate level of solubility.

The membrane binding element is preferably selected from: fatty acid derivatives such as fatty acyl groups; basic amino acid sequences; ligands of known integral membrane proteins; sequences derived from the complementarity-determining region of monoclonal antibodies raised against epitopes of membrane proteins; and membrane binding sequences identified through screening of random chemical or peptide libraries.

Examples of amino acid sequences derived from ligands of known integral membrane proteins include RGD-containing peptides such as GRGDSP (SEQ ID NO: 14) which are ligands for the α_{IIb}β_{3·} integrin of human platelet membranes. Another example is DGPSEILRGDFSS (SEQ ID NO: 15) derived from human fibrinogen alpha chain, which binds to the GpIIb/IIIa membrane protein in platelets.

Further examples of such sequences include those known to be involved in interactions between membrane proteins such as receptors and the major histocompatibility complex. An example of such a membrane protein ligand is the sequence GNEQSFRVDLRTLLRYA (SEQ ID NO: 16) which has been shown to bind to the major histocompatibility complex class 1 protein (MHC-1) with moderate affinity (L. Olsson et al, Proc. Natl .Acad.Sci.USA. 91, 9086-909, 1994). Yet further examples of such sequences employ a membrane insertive address specific for T-cells. Such sequence is derived from the known interaction of the transmembrane helix of the T-cell antigen receptor with CD3 (Nature Medicine 3, 84-88,1997). Examples are peptides containing the sequence GFRILLLKV (SEQ ID NO: 32) such as: SAAPSSGFRILLLKV (SEQ ID NO: 17) and AAPSVIGFRILLLKVAG (SEQ ID NO: 18). An example of a ligand for an integral membrane protein is the carbohydrate ligand Sialyl Lewis^{x} which has been identified as a ligand for the integral membrane protein ELAM-1 (M. L. Phillips et al, Science, 250, 1130-1132, 1990 & G. Walz et al, Ibid, 250, 1132-1135,1990). Sequences derived from the complementarity-determining regions of monoclonal antibodies raised against epitopes within membrane proteins (see, for example, J. W. Smith et al, J.Biol.Chem. 270, 30486-30490, 1995) are also suitable membrane binding elements, as are binding sequences from random chemical libraries such as those generated in a phage display format and selected by biopanning operations in vitro (G. F. Smith and J. K. Scott, Methods in Enzymology, 217H, 228-257,1993) or in vivo (R. Pasqualini & E. Ruoslahti, Nature, 380, 364-366, 1996). Optionally, conditional dissociation from the membrane may be incorporated into derivatives of the invention using mechanisms such as pH sensitivity (electrostatic switches), regulation through metal ion binding (using endogenous Ca²⁺, Zn²⁺ and incorporation of ion binding sites in membrane binding elements) and protease cleavage (e.g plasminolysis of lysine-rich membrane binding sequences to release and activate prourokinase).

In one embodiment, the membrane binding element may be a phospholipid which has been derivatised to increase its water-solubility. For example, the phospholipid may be derivatised with a hydrophilic polymer, such as polyethylene glycol (PEG), polyvinylpyrrolidone, dextran, or polysarcosine. Other suitable polymers would be apparent to a skilled person. However, it is preferred that the membrane binding element is not PEG.

The membrane binding element may comprise (or consist of) a glycosylphosphatidylinositol (GPI) anchor or an analogue thereof. Suitable GPI anchors and analogues are well known to those skilled in the art and are described, for example, in Paulick MG and Bertozzi CR (Biochemistry 47: 6991-7000, 2008). The carbohydrate portion of the GPI anchor may be comprised of any suitable saccharide monomers. Suitable saccharide monomers will be apparent to one skilled in the art as will the length of the carbohydrate portion. However, it is preferred that the membrane binding element is not a GPI anchor.

In an alternative embodiment, a membrane binding element may comprise (or consist of) a peptide which is capable of interacting with one or more components of the outer cell membranes of cells, for example, phospholipids. Preferably, the peptide is between 3 and 25 amino acids. More preferably, the peptide is between 4 and 20 amino acids. Preferably, the peptide is a hydrophilic peptide. In some embodiments a hydrophilic peptide comprises at least three charged amino acids. A charged amino acid may be lysine. In one embodiment, the peptide comprises between three and 8 lysine residues, preferably, L-lysine residues. A suitable hydrophilic peptide is shown as SEQ ID NO: 6. In one embodiment, a hydrophilic peptide may comprise (or consist of) a peptide sequence having at least 70% sequence identity to SEQ ID NO: 6. In one embodiment a hydrophilic peptide may comprise (or consist of) a peptide sequence having at least 80% or 90% sequence identity to SEQ ID NO: 6. Preferably, a hydrophilic peptide may comprise (or consist of) a peptide sequence having at least 95% sequence identity to SEQ ID NO: 6. More preferably, a hydrophilic peptide comprises (more preferably consists of) SEQ ID NO: 6. The cysteine residue comprised in the hydrophilic peptide may be activated cysteine, e.g. (S-2-pyridyldithio)-C-acid. Upon conjugation to the fusion polypeptide, the activated cysteine may undergo a chemical change such that it becomes a standard cysteine residue di-sulphide bonded to a corresponding cysteine residue of the fusion polypeptide.

Further suitable examples of peptides may include: DGPKKKKKKSPSKSSG (SEQ ID NO: 19); GSSKSPSKKKKKKPGD (SEQ ID NO: 20); SPSNETPKKKKKRFSFKKSG (SEQ ID NO: 21); DGPKKKKKKSPSKSSK (SEQ ID NO: 22); and SKDGKKKKKKSKTK (SEQ ID NO: 23).

A membrane binding element may comprise (or consist of) one or more hydrophobic groups that are capable of interacting with the lipid bilayer core of a cell membrane. Suitable groups are well known to those skilled in the art. In one embodiment, the one or more groups may be fatty acyl groups, such as myristoyl, palmitoyl, or stearoyl groups.

A fatty acid derivative herein may be a C₁₀₋₂₀ fatty acyl derivative of an amino C₂₋₆alkane thiol (optionally C-substituted) such as N-(2-myristoyl)aminoethanethiol or N-myristoyl L-cysteine.

Other examples of suitable hydrophobic groups include long-chain aliphatic amines and thiols, steroid and farnesyl derivatives. This approach is based on the structure and function of the myristoyl-electrostatic switch (MES) (Thelen M et al. Nature 351 : 320-2, 1991). In one embodiment, the one or more group is an isoprenoid group such as farnesyl and geranylgeranyl residues. Myristoyl (12 methylene units) is insufficiently large or hydrophobic to permit high affinity binding to membranes. Studies with myristoylated peptides (e.g. R. M. Peitzsch & S. McLaughlin, Biochemistry, 32, 10436-10443, 1993)) have shown that they have effective dissociation constants with model lipid systems of about 10⁻⁴ M and around 10 of the 12 methylene groups are buried in the lipid bilayer. Thus, aliphatic acyl groups with about 8 to 18 methylene units, preferably 10-14, are suitable membrane binding elements. Other examples of suitable fatty acid derivatives include long-chain (8-18, preferably 10-14 methylene) aliphatic amines and thiols, steroid and farnesyl derivatives.

Preferably a membrane binding element of the invention comprises an aliphatic acyl group, more preferably myristoyl or a derivative thereof.

Suitable examples of hydrophilic synthetic polymers include polyethyleneglycol (PEG), preferably α,ω functionalised derivatives, more preferably α-amino, ω-carboxy-PEG of molecular weight between 400 and 5000 daltons which are linked to the polypeptide for example by solid-phase synthesis methods (amino group derivatisation) or by thiol-interchange chemistry.

The membrane binding element may be a plurality of groups which are capable of interacting with the lipid bilayer core of a cell membrane. The compound of the invention may comprise one or more membrane binding elements. Preferably, the compound comprises one membrane binding element.

In one embodiment a membrane binding element comprises a combination of one or more hydrophobic groups capable of interacting with the lipid bilayer core of a cell membrane and a peptide capable of interacting with the lipid bilayer core of a cell membrane, such as a hydrophilic peptide described herein. Preferably said groups are located at, or near to, the N-terminal region of said peptide.

A membrane binding element may be one or more disclosed in WO 98/02454 or WO 2011/027175 (both of which are incorporated herein by reference) and the methodology of either of WO 98/02454 or WO 2011/027175 may be employed in preparing and conjugating a membrane binding element to a fusion polypeptide of the invention.

A membrane binding element may be conjugated to a cysteine residue or a lysine residue of the IL-15 activity-promoting sequence (preferably a cysteine residue). In a preferred embodiment a hydrophilic peptide portion of a membrane binding element is conjugated to a cysteine residue or a lysine residue of the IL-15 activity-promoting sequence (preferably a cysteine residue by way of a di-sulphide bond between a cysteine of the hydrophilic peptide portion of the membrane binding element and the IL-15 activity-promoting sequence).

Thus, in some embodiments, a fusion polypeptide conjugated to a membrane binding agent may comprise N-(α,εbis-myristoyllysine)SSKSPSKKDDKKPGDC linked to the polypeptide by a di-sulphide bond. The cysteine of the membrane binding agent (pre-conjugation) may be activated cysteine (e.g. thiopyridylated cysteine). The membrane binding agent may be one described in, and/or manufactured as per the teaching of, Hill A et al (2006), Blood, 107, 2131-2137, which is incorporated herein by reference in its entirety.

In one embodiment a polypeptide of the invention may have the following structure (SEQ ID NO: 7), which shows the presence of a di-sulphide bond between cysteine residues of the hydrophilic peptide portion of the membrane binding element and the IL-15 activity-promoting sequence:

```
          MNWVNVISDLKKIEDLIQSMHIDATLYTESDVHPSCKVTAMKCELLELQVIS
LESGDASIHDTVENLIILANNSLSSNGNVTESGCKECEELEEKNIKEFLQSFVHIVQ
MFINTSGSGSRGKSLTSKVPPTVQKPTTVNVPTTEVSPTSQKTTTHHHHHHC
                                                                                                      |
                                                                                                      S
                                                                                                        |
                                                                                                      S
                                                                                                      |
                    N-(α,εbis-myristoyllysine)SSKSPSKKDDKKPGDC
```

In one embodiment a polypeptide of the invention may have the following structure (SEQ ID NO: 29), which shows the presence of a di-sulphide bond between cysteine residues of the hydrophilic peptide portion of the membrane binding element and the IL-15 activity-promoting sequence:

```
          MNWVNVISDLKKIEDLIQSMHIDATLYTESDVHPSCKVTAMKCELLELQVIS
LESGDASIHDTVENLIILANNSLSSGNVTESGCKECEELEEKNIKEFLQSFVHIVQM
FINTSGSGSRGKSLTSKVPPTVQKPTTVNVPTTEVSPTSQKTTTHHHHHHC
                                                                                                    |
                                                                                                      S
                                                                                                      |
                                                                                                      S
                                                                                                      |
                    N-(α,εbis-myristoyllysine)SSKSPSKKDDKKPGDC
```

In one embodiment a polypeptide of the invention may have the following structure (SEQ ID NO: 13), which shows the presence of a di-sulphide bond between cysteine residues of the hydrophilic peptide portion of the membrane binding element and the IL-15 activity-promoting sequence:

```
            MNWVNVISDLKKIEDLIQSMHIDATLYTESDVHPSCKVTAMKCF
LLELQVISLESGDASIHDTVENLIILANNSLSSGNVTESGCKECEELEEK
                          NIKEFLQSFVHIVQMFINTSGSGSHHHHHHC
                                                                                      |
                                                                                        S
                                                                                        |
                                                                                        S
                                                                                        |
      N-(α,εbis-myristoyllysine)SSKSPSKKDDKKPGDC
```

A fusion polypeptide comprising a membrane binding element may comprise (or consist of) a polypeptide sequence having at least 70% sequence identity to SEQ ID NO: 7. In one embodiment a fusion polypeptide comprising a membrane binding element may comprise (or consist of) a polypeptide sequence having at least 80% or 90% sequence identity to SEQ ID NO: 7. Preferably, a fusion polypeptide comprising a membrane binding element may comprise (or consist of) a polypeptide sequence having at least SEQ ID NO: 7. More preferably, a fusion polypeptide comprising a membrane binding element comprises (more preferably consists of) SEQ ID NO: 7.

A fusion polypeptide comprising a membrane binding element may comprise (or consist of) a polypeptide sequence having at least 70% sequence identity to SEQ ID NO: 13. In one embodiment a fusion polypeptide comprising a membrane binding element may comprise (or consist of) a polypeptide sequence having at least 80% or 90% sequence identity to SEQ ID NO: 13. Preferably, a fusion polypeptide comprising a membrane binding element may comprise (or consist of) a polypeptide sequence having at least SEQ ID NO: 13. More preferably, a fusion polypeptide comprising a membrane binding element comprises (more preferably consists of) SEQ ID NO: 13.

A fusion polypeptide comprising a membrane binding element may comprise (or consist of) a polypeptide sequence having at least 70% sequence identity to SEQ ID NO: 29. In one embodiment a fusion polypeptide comprising a membrane binding element may comprise (or consist of) a polypeptide sequence having at least 80% or 90% sequence identity to SEQ ID NO: 29. Preferably, a fusion polypeptide comprising a membrane binding element may comprise (or consist of) a polypeptide sequence having at least SEQ ID NO: 29. More preferably, a fusion polypeptide comprising a membrane binding element comprises (more preferably consists of) SEQ ID NO: 29.

While the fusion polypeptide comprising a membrane binding element may comprise (or consist of) SEQ ID NO: 7, 13 or 29, a fusion polypeptide comprising (or consisting of) SEQ ID NO: 7 is preferred.

In some embodiments the cysteine(s) involved in the conjugation of the fusion polypeptide to the membrane binding element are modified cysteine residues (preferably standard cysteine residues). Modified cysteine residues may include an amide form of cysteine (cysteine amide).

The present invention also provides nucleic acids encoding a fusion polypeptide of the invention (i.e. a protein component of a fusion polypeptide of the invention). The nucleic acid is preferably DNA.

A nucleic acid of the invention may be comprised in a vector for expression in a host cell. Thus, the invention also provides vectors and host cells comprising a nucleic acid of the invention. The vectors may comprise a promoter operably linked to a nucleic acid of the invention and may further comprise a terminator.

In one embodiment a nucleic acid encoding a fusion polypeptide of the invention comprises (or consists of) a nucleotide sequence having at least 70% sequence identity to SEQ ID NO: 8. In one embodiment a nucleic acid encoding a fusion polypeptide of the invention comprises (or consists of) a nucleotide sequence having at least 80% or 90% sequence identity to SEQ ID NO: 8. Preferably, a nucleic acid encoding a fusion polypeptide of the invention comprises (or consists of) a nucleotide sequence having at least 95% sequence identity to SEQ ID NO: 8. More preferably, a nucleic acid encoding a fusion polypeptide of the invention comprises (more preferably consists of) SEQ ID NO: 8.

In one embodiment a nucleic acid encoding a fusion polypeptide of the invention comprises (or consists of) a nucleotide sequence having at least 70% sequence identity to SEQ ID NO: 24. In one embodiment a nucleic acid encoding a fusion polypeptide of the invention comprises (or consists of) a nucleotide sequence having at least 80% or 90% sequence identity to SEQ ID NO: 24. Preferably, a nucleic acid encoding a fusion polypeptide of the invention comprises (or consists of) a nucleotide sequence having at least 95% sequence identity to SEQ ID NO: 24. More preferably, a nucleic acid encoding a fusion polypeptide of the invention comprises (more preferably consists of) SEQ ID NO: 24.

Any suitable host cell may be employed for production of a fusion polypeptide of the invention. A host cell may be a eukaryotic or prokaryotic host cell. Suitable eukaryotic cells may include mammalian cells (e.g. HEK293 cells or HeLa cells), yeast cells (e.g. Saccharomyces cerevisiae or Pichia pastoris) or insect cells (e.g. baculovirus-infected insect cells).

In one embodiment a host cell is a prokaryotic host cell, e.g. of the genus Escherichia or Bacillus (e.g. Bacillus subtilis). Preferably, a host cell is an Escherichia coli host cell.

In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

In another preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

IPTG refers to Isopropyl β-D-1-thiogalactopyranoside.

The nucleic acid molecules of the invention may be made using any suitable process known in the art. In one embodiment, the nucleic acid molecules may be made using chemical synthesis techniques. Alternatively, the nucleic acid molecules of the invention may be made using molecular biology techniques.

The DNA construct of the present invention may be designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

In one aspect, the invention is directed to a method for producing a fusion polypeptide, the method comprising:
a. expressing the nucleic acid sequence encoding a fusion polypeptide of the invention in a host cell; and
b. isolating the fusion polypeptide.

An isolated fusion polypeptide may be free from alternative polypeptides or cellular matter, e.g. substantially free from any alternative polypeptides or cellular matter. In other words, a fusion polypeptide may be considered “isolated” when the fusion polypeptide of the invention constitutes at least 90% of the total polypeptides present, preferably when the fusion polypeptide of the invention constitutes at least 95%, 98% or 99% (more preferably at least 99.9%) of the total polypeptides present. Isolating can be achieved using any suitable methods known in the art such as any suitable purification methods, e.g. chromatographic methods. Suitable methods may include affinity chromatography, ion exchange (e.g. cation or anion exchange) chromatography and immunoaffinity chromatography. Preferably purification is by way of metal-chelate chromatography, more preferably nickel-chelate chromatography. In some embodiments the polypeptides of the invention may further comprise a tag to aid in purification, such as a His-tag, which may be subsequently removed, e.g. by way of a cleavage site, such as a TEV cleavage site, engineered between the tag and polypeptide.

In a related aspect, the invention provides a fusion polypeptide obtainable by a method of the invention.

The term “obtainable” as used herein also encompasses the term “obtained”. In one embodiment the term “obtainable” means obtained.

A fusion polypeptide of the invention may be formulated in any suitable manner. Thus, in one embodiment, the invention provides a pharmaceutical composition comprising a fusion polypeptide of the invention and a pharmaceutically acceptable carrier, excipient, adjuvant, and/or salt. The term “pharmaceutically acceptable carrier, excipient, adjuvant, and/or salt” as used herein means a carrier, excipient, adjuvant, and/or salt that can be administered to a subject without causing harm to said subject. For example, a carrier, excipient, adjuvant, and/or salt that is suitable for intratumoural, intravenous, intra-arterial, intraperitoneal, intrathecal intramuscular, and/or subcutaneous administration. In one embodiment a pharmaceutically acceptable carrier, excipient, adjuvant, and/or salt is an injectable carrier, excipient, adjuvant, and/or salt, such as a sterile physiological saline solution.

Pharmaceutically acceptable excipients that may be used in the pharmaceutical composition of the invention include, but are not limited to serum proteins, such as human serum albumin, buffer substances such as phosphates, glycerine, sorbic acid, potassium sorbate, partial glyceride mixtures of saturated vegetable fatty acids, water, salts or electrolytes, disodium hydrogen phosphate, potassium hydrogen phosphate, and sodium chloride. The pharmaceutical compositions of this invention may contain any conventional non-toxic pharmaceutically-acceptable carriers or vehicles. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant. Preferably, the fusion polypeptides of the invention are present in an aqueous solution.

Other pharmaceutically acceptable additives which may be added to the composition are well known to those skilled in the art.

In one aspect the invention also provides a kit comprising: a fusion polypeptide or a pharmaceutical composition of the invention; and instructions for use of the same. Suitably, the instructions may be for the use of the same in treating cancer as described herein. In some embodiments the instructions also detail an appropriate dosage regimen (e.g. as described herein). In one embodiment the instructions are for use of said kit in treating prostate cancer.

A fusion polypeptide of the invention is particularly suitable for use in treating cancer. Thus, in one aspect the present invention provides a fusion polypeptide for use in treating cancer. The invention also provides a related use of a fusion polypeptide of the invention in the manufacture of medicament for treating cancer, and methods of treating cancer comprising administering a fusion polypeptide of the invention to a subject. Analogous uses/methods of the pharmaceutical composition (or other contemplated formulations) are also provided. Analogous uses/methods of the kit are also provided.

A fusion polypeptide of the invention may inhibit growth, proliferation and/or metastasis of a cancer cell. For example, a fusion polypeptide of the invention may eradicate cancer cells, inhibit cancer cell proliferation, and/or reduce the size of a cancer.

A cancer for treatment is preferably not a haematological cancer, such as leukaemia, lymphoma and/or multiple myeloma.

In one embodiment a cancer is a solid tumour cancer, e.g. a carcinoma or a sarcoma.

A solid tumour cancer may be a sarcoma, such as osteosarcoma or osteogenic sarcoma (bone), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), or mesenchymous or mixed mesodermal tumor (mixed connective tissue types).

Preferably, a cancer is a carcinoma. A carcinoma may be an adenocarcinoma (which develops in an organ or gland) or a squamous cell carcinoma (which originates from squamous epilthelium). Preferably, a carcinoma is an adenocarcinoma.

Alternatively or additionally, a solid tumour cancer may be of a mixed type containing components from one or more different cancer category. Some examples of mixed type cancers include adenosquamous carcinomas, mixed mesodermal tumours, carcinosarcomas, and teratocarcinomas.

A cancer (e.g. solid tumour cancer) treated in accordance with the present invention may be one or more selected from: prostate cancer, colon cancer, breast cancer, lung cancer, skin cancer, liver cancer, bone cancer, ovarian cancer, pancreatic cancer, brain cancer, head cancer, neck cancer, lymphoma, and neuronal cancer.

In a particularly preferred embodiment the cancer is prostate cancer. The prostate cancer may be ductal prostate cancer or acinar prostate cancer, preferably ductal prostate cancer.

A fusion polypeptide or pharmaceutical composition may be administered to a subject in a therapeutically effective amount or a prophylactically effective amount.

The terms “subject” and “patient” are used synonymously herein. The “subject” may be a mammalian subject, for example a human, a companion animal (e.g. a pet such as dogs, cats, and rabbits), livestock (e.g. pigs, sheep, cattle, and goats), and horses. Preferably, a “subject” is a human subject.

The term “treat” or “treating” as used herein encompasses prophylactic treatment (e.g. to prevent onset of a disease) as well as corrective treatment (treatment of a subject already suffering from a disease). Preferably “treat” or “treating” as used herein means corrective treatment.

The term “treat” or “treating” as used herein refers to the disorder and/or a symptom thereof.

A “therapeutically effective amount” is any amount of the fusion polypeptide or pharmaceutical composition of the invention, which when administered alone or in combination to a subject for treating cancer (or a symptom thereof) is sufficient to effect such treatment of the disorder, or symptom thereof.

A “prophylactically effective amount” is any amount of the fusion polypeptide or pharmaceutical composition of the invention that, when administered alone or in combination to a subject inhibits or delays the onset or reoccurrence of cancer (or a symptom thereof). In some embodiments, the prophylactically effective amount prevents the onset or reoccurrence of cancer entirely. “Inhibiting” the onset means either lessening the likelihood of onset of cancer (or symptom thereof), or preventing the onset entirely.

An appropriate dosage range is one that produces the desired therapeutic effect (e.g. wherein the fusion polypeptide or pharmaceutical composition is dosed in a therapeutically or prophylactically effective amount).

A typical treatment regimen may include administering a fusion polypeptide or pharmaceutical composition of the invention at a dosage of up to 1 mg of fusion polypeptide to the subject (e.g. intravenously or subcutaneously), for example at a dosage of 0.1-1 mg, e.g. 0.2-0.5 mg.

A subject for treatment may be dosed once, twice, three times, four times, five times, or six times per week. Alternatively, a subject may be dosed daily (e.g. once or twice daily). In other embodiments a subject may be dosed once weekly or bi-weekly. Preferably, the subject may be dosed once every two weeks.

The skilled person will appreciate that the dose can be tailored based on the needs of the subject, and efficacy of the medicament. For example, where the medicament is highly efficacious, the dose may be lowered.

The treatment term can be varied based on the response of the subject to the treatment, and/or the type and/or severity of the cancer.

Administration may be by any suitable technique or route, including but not limited to intratumourally, intravenously, intra-arterially, intraperitoneally, intrathecally, intramuscularly, and/or subcutaneously. While different methods of administration are contemplated by the present invention, it is particularly preferred that a fusion polypeptide of the invention is administered intratumourally. Such, intratumoural administration may be achieved by intratumoural injection.

A subject may be treated with a fusion polypeptide or pharmaceutical composition of the invention in combination with a different cancer therapeutic, such as a chemotherapy agent or immunotherapy agent. In other words, a fusion polypeptide or pharmaceutical composition may be an adjuvant therapy.

Embodiments related to the various fusion polypeptides of the invention are intended to be applied equally to the methods, uses, kits or pharmaceutical compositions, and vice versa.

### SEQUENCE HOMOLOGY

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position—Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### CONSERVATIVE AMINO ACID SUBSTITUTIONS

Basic: arginine
lysine
histidine

Acidic: glutamic acid
aspartic acid

Polar: glutamine
asparagine

Hydrophobic: leucine
isoleucine
valine

Aromatic: phenylalanine
tryptophan
tyrosine

Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; WIodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a fusion polypeptide” includes a plurality of such candidate agents and reference to “the fusion polypeptide” includes reference to one or more fusion polypeptides and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows: (A) the ability of various Th1 cytokines (compared to phosphate buffered saline (PBS) control) to expand and activate Natural Killer (NK) cells and CD8 T cells in co-cultures of peripheral blood mononuclear cells (PBMCs) and prostate cancer cells (PC3 and LNCaP); and (B) NK and CD8 T cell cytotoxic capabilities by way of perforin expression and apoptotic and necrotic cell death of tumour cells by way of Annexin-FITC and propidium iodide (PI) staining.

FIG. 2 shows the activity of modified IL-15 (containing an activity-promoting sequence) compared to unmodified wild-type IL-15 in a CTLL-2 assay as described in the Examples.

FIG. 3 shows visualisation of tailed IL-15 by gel electrophoresis followed by silver nitrate staining, western blot analysis and UV light visualisation of fluorescently labelled tail compound PTL3146. The band circled represents the main tailed protein moiety. Lane 4 contains the newly prepared modified IL-15, which is pure on silver stain and anti-IL-15 western blot. Lane key: 1=marker; 2, 4=modified IL-15; and 3=membrane-anchored modified IL-15 with a FAM-labelled tail.

FIG. 4 shows cell membrane binding of membrane-anchored modified IL-15 (“tailed Il-15”) and modified IL-15 (“untailed IL-15”) by flow cytometry on: (A) Jurkat cells after 30 minutes and 24 hours; and (B) sheep red blood cells.

FIG. 5 shows the activity of modified IL-15 (“untailed”) vs. membrane-anchored modified IL-15 (“tailed”) and wild-type unmodified IL-15. Proliferation was measured by a CTLL-2 assay as described in the Examples at an absorbance of 490 nm for n=3 experiments.

FIG. 6 shows comparison of NK expansion in a PBMC population treated with IL-2 (100 units per ml), wild-type IL-15, modified IL-15 (“untailed IL-15”) and membrane-anchored modified IL-15 (“tailed IL-15”) (2.5 ng/ml each). A shows representative dot blots from Flow cytometry analysis. Top left quadrant on the dot blots represents NK cells (CD56+CD3−). B shows a graph (human PBMCs) showing expansion of human NK cells by the tested IL-15 polypeptides. Control=PBS only.

FIG. 7 shows killing of PC3 cells co-cultured with human NK cells in the presence of IL-2, wild-type IL-15 (IL-15 pep.), modified IL-15 (“untailed IL-15”) and membrane-anchored modified IL-15 (“tailed IL-15”). Cell killing is represented by positive staining of the cells with propidium iodide (Pl). n=2, *p<0.05 by one-way ANOVA and post-hoc test Newman-Keuls. Control=PBS only.

FIG. 8 shows the effect of IL-15 on growth of TRAMP-C2 prostate tumour xenografts. Mice with TRAMP-C2 tumours of approximately 100 mm³ were injected intratumourally vehicle (100 μl PBS, n=10), 10 μg untailed IL-15 (n=10) or tailed IL-15 (n=10), or intraperitoneally with untailed IL-15 (n=6) at days 0 and 3. (A) Tumour volumes up to day 14 post-treatment. (B) Survival curves of treated mice post-treatment. Survival endpoint was when tumours reach a maximum diameter of 15 mm. No side effects were caused by any of the treatments (*p<0.05, **p<0.01, ***P<0.001 by two-way ANOVA with Dunnett multiple comparisons post-test).

FIG. 9 shows ex vivo histopathological assessment of TRAMP-C2 prostate tumours. Tumours were excised at experimental endpoints, snap frozen and subsequently sectioned at 10 μm sections. (a) Composite images of H&E-stained sections indicating necrotic regions and magnified regions of the same images. (b) Composite images from sections stained with NK1.1 (NK cell) and CD3 antibodies. (c) Composite images from sections stained CD8 and KLRA1 (NK cell) antibodies. (d) Composite images from sections stained with CD4 antibody. Nuclei in all fluorescent sections were stained with DAPI.

FIG. 10 shows a quantitation of the “vehicle” and “tailed IL-15” histopathological assessment of FIG. 9: a) Necrosis b) CD8+ staining, c) CD4+ staining, d) CD3+ staining and e) NK1.1 (NK cell) staining. Quantitation was based on results obtained from at least 6 animals in each group.

FIG. 11 shows proliferation of CTLL-2 cells after incubation with varying concentrations of wild-type IL-15 and modified IL-15 polypeptides SEQ ID NO: 28, SEQ ID NO: 10, and SEQ ID NO: 12 as measured by IL-15 ELISA. Proliferation was measured by MTS assay at an absorbance of 490 nm. N=2.*=p<0.05 by T-Test for SEQ ID NO: 28 vs SEQ ID NO: 10. SEQ ID NO: 28 is significantly more active vs SEQ ID NO: 12 and wild-type IL-15 at all concentrations (p<0.05 by 1 way ANOVA and Tukey test).

FIG. 12 shows binding of FITC labelled SEQ ID NO: 28 and wild-type IL-15 to CTLL-2 cells as analysed by flow cytometry using a FACs Calibur (BD Biosciences).

SEQUENCE LISTING
Where an initial Met amino acid residue or a corresponding initial
codon is indicated in any of the following SEQ ID NOs, said residue/codon
is optional.
(Full-Length Interleukin-15)
SEQ ID NO: 1
MRISKPHLRSISIQCYLCLLLNSHFLTEAGIHVFILGCFSAGLPKTEANWVNVISDLKKI
EDLIQSMHIDATLYTESDVHPSCKVTAMKCFLLELQVISLESGDASIHDTVENLIILANN
SLSSNGNVTESGCKECEELEEKNIKEFLQSFVHIVQMFINTS
(Mature Interleukin-15 - Amino Acids 49-162)
SEQ ID NO: 2
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFINTS
(Mature Interleukin-15)
SEQ ID NO: 3
MNWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFINTS
(Activity-Promoting Seguence)
SEQ ID NO: 4
GSGSRGKSLTSKVPPTVQKPTTVNVPTTEVSPTSQKTTTHHHHHHC
(Fusion Polypeptide)
SEQ ID NO: 5
MNWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFINTSGSGSRGKSLTSKVP
PTVQKPTTVNVPTTEVSPTSQKTTTHHHHHHC
(Hydrophilic Peptide)
SEQ ID NO: 6
SSKSPSKKDDKKPGDC
(Fusion Polypeptide Comprising a Membrane Binding Element)
SEQ ID NO: 7
MNWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFINTSGSGSRGKSLTSKVP
PTVQKPTTVNVPTTEVSPTSQKTTTHHHHHHC*
N-(α,ε bis-myristoyl lysine)SSKSPSKKDDKKPGDC*
*indicates the location of the di-sulphide bond between the activity-promoting
peptide and membrane binding element.
(Nucleic Acid Seguence Encoding SEQ ID NO: 28)
SEQ ID NO: 8
AACTGGGTGA ACGTTATCTC GGACCTGAAA AAAATCGAAG ACCTGATCCA AAGCATGCAC
ATTGACGCTA CGCTGTATAC GGAAAGCGAT GTGCATCCGT CGTGCAAAGT TACCGCGATG
AAATGTTTTC TGCTGGAACT GCAGGTCATT TCGCTGGAAA GCGGCGATGC GAGTATCCAC
GACACCGTTG AAAACCTGAT TATCCTGGCC AACAATTCCC TGAGCTCTGG CAATGTGACG
GAATCAGGTT GCAAAGAATG TGAAGAACTG GAAGAGAAAA ACATCAAAGA ATTCCTGCAG
TCTTTCGTCC ATATTGTGCA AATGTTCATC AATACGAGTG GCTCCGGTTC ACGTGGTAAA
TCTCTGACCA GTAAAGTTCC GCCGACGGTC CAAAAACCGA CCACGGTGAA CGTTCCGACC
ACCGAAGTCT CTCCGACCAG TCAGAAAACC ACCACCCACC ATCACCATCA TCATTGC
(Activity-Promoting Seguence 2)
SEQ ID NO: 9
GSGSHHHHHHC
(Fusion Polypeptide 2)
SEQ ID NO: 10
MNWVNVISDLKKIEDLIQSMHIDATLYTESDVHPSCKVTAMKCFLLELQVISLESGDASIHDTVENLIILANNSL
SSGNVTESGCKECEELEEKNIKEFLQSFVHIVQMFINTSGSGSHHHHHHC
(Comparative Fusion Sequence)
SEQ ID NO: 11
GSGSRGKSLTSKVPPTVQKPTTVNVPTTEVSPTSQKTTTKTTTPNAQATRSTPVSRTTKHHHHHHHC
(Comparative Fusion Polypeptide)
SEQ ID NO: 12
MNWVNVISDLKKIEDLIQSMHIDATLYTESDVHPSCKVTAMKCFLLELQVISLESGDASIHDTVENLIILANNSL
SSGNVTESGCKECEELEEKNIKEFLQSFVHIVQMFINTSGSGSRGKSLTSKVPPTVQKPTTVNVPTTEVSPTSQK
TTTKTTTPNAQATRSTPVSRTTKHHHHHHHC
(Fusion Polypeptide 2 Comprisinq a Membrane Bindinq Element)
SEQ ID NO: 13
MNWVNVISDLKKIEDLIQSMHIDATLYTESDVHPSCKVTAMKCFLLELQVISLESGDASIHDTVENLIILANNSL
SSGNVTESGCKECEELEEKNIKEFLQSFVHIVQMFINTSGSGSHHHHHHC*
N-(α,ε bis-myristoyl lysine) SSKSPSKKDDKKPGDC*
*indicates the location of the di-sulphide bond between the activity-promoting
peptide and membrane binding element.
(Nucleic Acid Sequence Encodinq SEQ ID NO: 28 plus Met)
SEQ ID NO: 24
ATGAACTGGGTGA ACGTTATCTC GGACCTGAAA AAAATCGAAG ACCTGATCCA AAGCATGCAC
ATTGACGCTA CGCTGTATAC GGAAAGCGAT GTGCATCCGT CGTGCAAAGT TACCGCGATG
AAATGTTTTC TGCTGGAACT GCAGGTCATT TCGCTGGAAA GCGGCGATGC GAGTATCCAC
GACACCGTTG AAAACCTGAT TATCCTGGCC AACAATTCCC TGAGCTCTGG CAATGTGACG
GAATCAGGTT GCAAAGAATG TGAAGAACTG GAAGAGAAAA ACATCAAAGA ATTCCTGCAG
TCTTTCGTCC ATATTGTGCA AATGTTCATC AATACGAGTG GCTCCGGTTC ACGTGGTAAA
TCTCTGACCA GTAAAGTTCC GCCGACGGTC CAAAAACCGA CCACGGTGAA CGTTCCGACC
ACCGAAGTCT CTCCGACCAG TCAGAAAACC ACCACCCACC ATCACCATCA TCATTGC
(Full-Lenqth Interleukin-15 Variant)
SEQ ID NO: 25
MRISKPHLRSISIQCYLCLLLNSHFLTEAGIHVFILGCFSAGLPKTEANWVNVISDLKKI
EDLIQSMHIDATLYTESDVHPSCKVTAMKCFLLELQVISLESGDASIHDTVENLIILANN
SLSSGNVTESGCKECEELEEKNIKEFLQSFVHIVQMFINTS
(Mature Interleukin-15 - Amino Acids 49-162 Variant)
SEQ ID NO: 26
NWVNVISDLKKIEDLIQSMHIDATLYTESDVHPSCKVTAMKCFLLELQVISLESGDASIH
DTVENLIILANNSLSSGNVTESGCKECEELEEKNIKEFLQSFVHIVQMFINTS
(Mature Interleukin-15 Variant)
SEQ ID NO: 27
MNWVNVISDLKKIEDLIQSMHIDATLYTESDVHPSCKVTAMKCFLLELQVISLESGDASIH
DTVENLIILANNSLSSGNVTESGCKECEELEEKNIKEFLQSFVHIVQMFINTS
(Fusion Polypeptide)
SEQ ID NO: 28
MNWVNVISDLKKIEDLIQSMHIDATLYTESDVHPSCKVTAMKCFLLELQVISLESGDASIH
DTVENLIILANNSLSSGNVTESGCKECEELEEKNIKEFLQSFVHIVQMFINTSGSGSRGKSLTSKVPP
TVQKPTTVNVPTTEVSPTSQKTTTHHHHHHC
(Fusion Polypeptide Comprising a Membrane Binding Element)
SEQ ID NO: 29
MNWVNVISDLKKIEDLIQSMHIDATLYTESDVHPSCKVTAMKCFLLELQVISLESGDASIH
DTVENLIILANNSLSSGNVTESGCKECEELEEKNIKEFLQSFVHIVQMFINTSGSGSRGKSLTSKVPP
TVQKPTTVNVPTTEVSPTSQKTTTHHHHHHC*
N-(α,ε bis-myristoyl lysine) SSKSPSKKDDKKPGDC*
*indicates the location of the di-sulphide bond between the activity-promoting
peptide and membrane binding element.
(Membrane Binding Element)
SEQ ID NO: 31
N-(α, εbis-myristoyllysine) SSKSPSKKDDKKPGDC*
*indicates the location of the di-sulphide bond between the activity-promoting
peptide and membrane binding element.

### EXAMPLES

### Example 1

Cytokine Selection

Non-adherent PBMCs were cultured for 7 days with irradiated PC3 cells in an 8:1 ratio and stimulated with IL-2, IFN-gamma, IL-12, IL-15 or IL-21 used at ED₅₀ doses (25 ng/ml for IFN gamma, IL-12, IL-15 and IL-21, and 100 units/ml for IL-2). Expansion of effector cells was measured using anti-CD3, CD56, CD4, CD8, CD25 and FOXP3 antibodies. Results were analysed on a FACSCalibur. NK and CD8 T cell cytotoxic capabilities were assessed by measuring perforin. Apoptotic and necrotic cell death was assessed by staining tumour cells with Annexin-FITC, and propidium iodide using an Annexin/PI kit (Invitrogen).

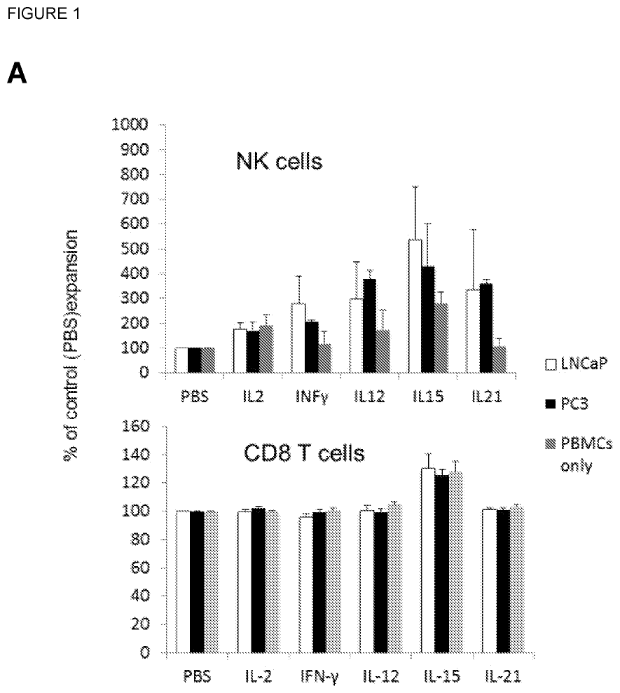

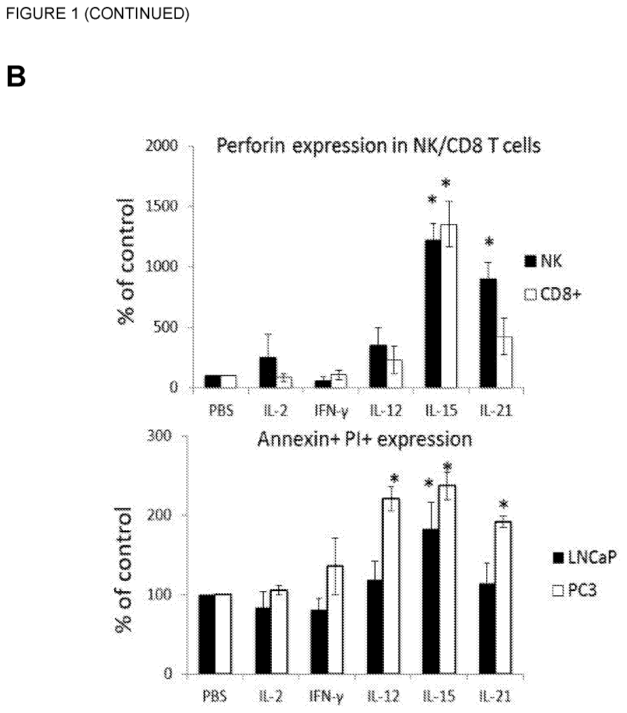

The results showed that IL-15 is superior to other selected Th1 cytokines at activating and expanding NK, NKT and CD8 T cells in co-cultures of PBMCs and prostate cancer cells (FIG. 1). IL-15 was therefore selected for further characterisation and testing as an appropriate therapeutic for treating cancer.

### Example 2

Modified IL-15 (Fusion Polypeptide of the Invention)

The mature form of human IL-15 was fused to an extended C-terminal sequence shown as SEQ ID NO: 4 and recombinantly expressed in E. coli.

The modified form of IL-15 was tested using a CTLL-2 assay (Soman G, Yang X, Jiang H, et al. MTS dye based colorimetric CTLL-2 cell proliferation assay for product release and stability monitoring of Interleukin-15: Assay qualification, standardization and statistical analysis. Journal of immunological methods. 2009; 348(1-2):83-94). Briefly, CTLL-2 cells (a mouse CD8 T cell line) were grown in the presence of IL-15. Said cells only proliferate when exposed to Interleukin-2 or Interleukin-15. The cells were cultured at a concentration of 1×10⁴ cells/ml in 96 well plates for 48 hours in the presence of a range of doses of IL-15. At the 48 hour time point cells were stained with MTS (5-[3-(carboxymethoxy)phenyl]-3-(4,5-dimethyl-2-thiazolyl)-2-(4-sulfophenyl)-2H-tetrazolium inner salt), which correlated with the numbers of cells detected.

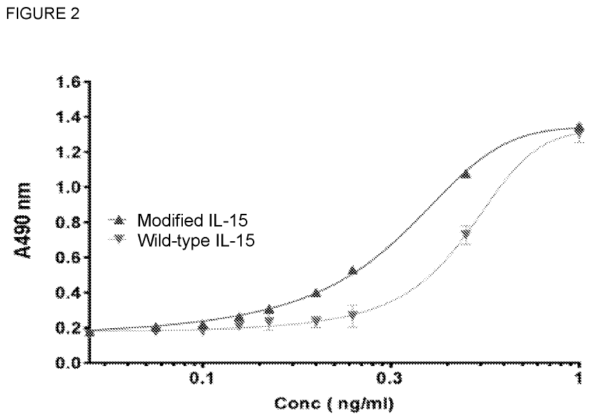

Surprisingly, the modified form of IL-15 was found to have improved activity when compared to the unmodified wild-type IL-15 (see FIG. 2). Thus, the extended C-terminal sequence was found to promote IL-15 activity. Without wishing to be bound by theory, it is believed that the IL-15 activity-promoting sequence may stabilise the interaction of IL-15 with its receptor, thus stimulating CLL-2 cell proliferation.

### Example 3

Preparation of Membrane-Anchored IL-15

In an attempt to further improve the therapeutic utility of the modified IL-15, it was decided to introduce an additional modification to localise the polypeptide to cell membranes. To achieve this, cytotopic modification was employed. This procedure employs the use of a hydrophobic membrane-insertive myristoyl group, linked by hydrophilically charged amino-acids and a C-terminal-activated disulphide (the combination of these is referred to as the “tail”), which is attached to a protein or peptide directly (through free thiol groups) or indirectly (through thiolated lysine residues) in the latter structure. The reaction creates stable amphipathic compounds which can be tethered to the phosphatidyl-serine rich regions of cell membranes. The tethering process is driven by two non-covalent interactions: one hydrophobic (myristoyl) and one electrostatic (based on lysine residues). Therefore, such agents can localise in any tissue into which they are injected.

The modified IL-15 of Example 2 was conjugated to a tail compound, PTL3146 N-(α,ε bis-myristoyl lysine) SSKSPSKKDDKKPGD(S-2-pyridyldithio)-C-acid (SEQ ID NO: 30) (MW of 3 KDa) using a standard procedure: after a mild reduction step (incubation with 100 μM TCEP overnight at room temperature), modified IL-15 was incubated with PTL3146 for an hour at room temperature at a 3:1 molar ratio, followed by overnight dialysis in 1 litre of PBS at 4° C. to remove excess tail.

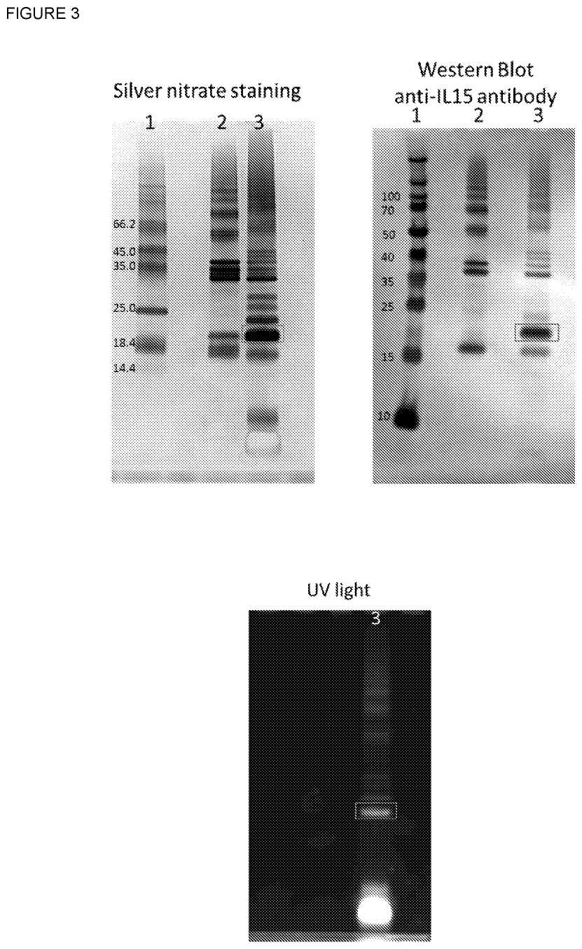

The attachment of the tail to modified IL-15 was confirmed using gel electrophoresis of the untailed and tailed protein using a tail labelled with the fluorophore FAM (Carboxyfluorescein), and western blot analysis using an antibody to IL-15 that recognises active protein (FIG. 3).

### Example 4

Confirmation of Binding of Membrane-Anchored IL-15 to Cell Membranes

To test the ability of the membrane-anchored IL-15 (tailed IL-15) of Example 3 to bind to cell membranes, assays using sheep red blood erythrocytes or Jurkat cells were employed. These cell types were chosen as they do not have receptors or proteins that can bind IL-15. Binding of tailed IL-15 to these cells was assessed by flow cytometric analysis using a Phycoerythrin (PE) labelled antibody to IL-15. Briefly, the relevant IL-15 polypeptides were incubated with either Jurkat cells or Sheep Red Blood Cells (Cat. Number ABIN770405, antibodies-online). Cells were centrifuged and resuspended in 4 ml of PBS containing 2% FCS to a final concentration of 2×10⁶ cells/ml. After dilution, cells were centrifuged at 1800 rpm for 5 minutes at room temperature and the supernatant was discarded. Cells were incubated at room temperature for 20 minutes with 2 μg of either tailed or untailed IL-15. Unbound IL-15 was removed by washing the cells with PBS containing 2% FCS followed by a centrifugation at 1800 rpm for 5 minutes at room temperature. Supernatant was removed and cells were incubated in the dark for 20 minutes at 4° C. with 2 μl of mouse anti-human IL15 PE conjugated antibody (Cat. Number IC2471P, R&D Systems). The washing step was repeated twice, and cells were resuspended in 400 μl PBS containing 2% FCS and analysed by Flow Cytometry.

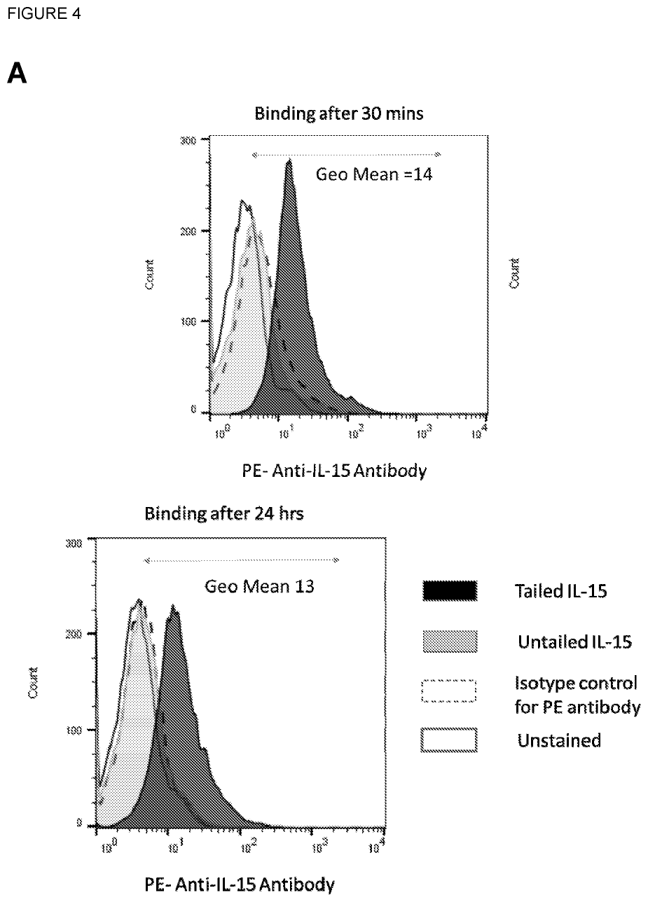

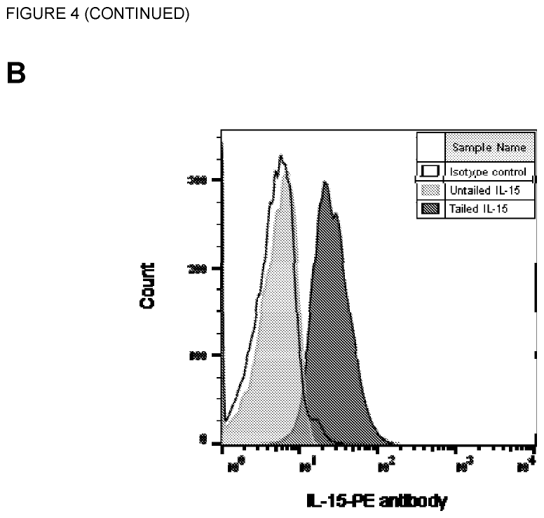

FIG. 4 shows that no binding was seen with untailed IL-15 either on sheep red blood cells (b) or Jurkat cells (a). In contrast membrane-anchored IL-15 (tailed IL-15) exhibited high levels of cell binding, with similar results obtained with 30 min or 24 h incubation of tailed IL-15 on Jurkat cells (b) showing that it can be retained on cell membranes through the tail portion of the molecule for a significant period of time. Internalisation is therefore slow allowing significant cell-surface binding and presentation for activity.

### Example 5

Study of the Activity of Membrane-Anchored IL-15 In Vitro

The activity of the membrane-anchored modified IL-15 of Example 3 (tailed IL-15) was compared to the non-anchored modified IL-15 of Example 2 (untailed IL-15) and unmodified wild-type control IL-15 using a CTLL2 assay:

a) murine CTLL-2 cells (LGC standards, UK [cat no. ATCC® TIB-214™]) were cultured at a concentration of 5×10⁵ cells/ml in 96 well plates (5×10⁴ cells per well in a volume of 100 ul) for 72 hours in the presence of tailed IL-15, untailed IL-15, or antibody only, or in the absence of any IL-15 polypeptide or antibody (unstained) at 37° C.;

b) cells were incubated with MTS (5-[3-(carboxymethoxy)phenyl]-3-(4,5-dimethyl-2-thiazolyI)-2-(4-sulfophenyl)-2H-tetrazolium inner salt) (Promega [CellTiter 96® AQueous One Solution Cell Proliferation Assay]) for 3-4 hours (at the 72 hour time point); and

c) the number of cells was quantified by colorimetry at an absorbance of 490 nm.

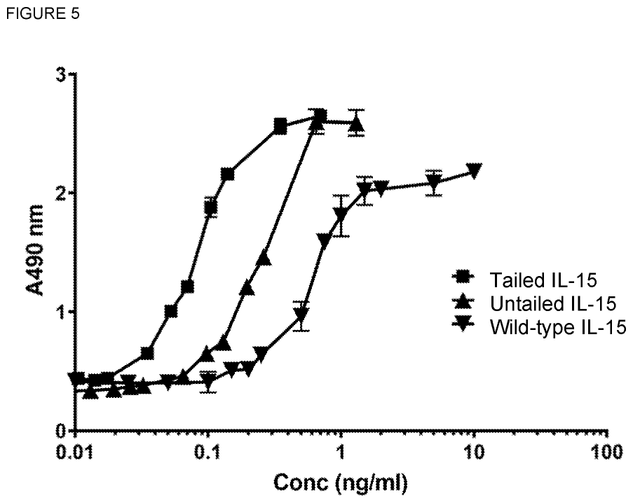

FIG. 5 shows that, consistent with the results of Example 2, the non-anchored modified IL-15 (untailed) was significantly more active than wild-type IL-15. However, membrane-anchored modified IL-15 (tailed) was, advantageously, more active than either the untailed or wild-type.

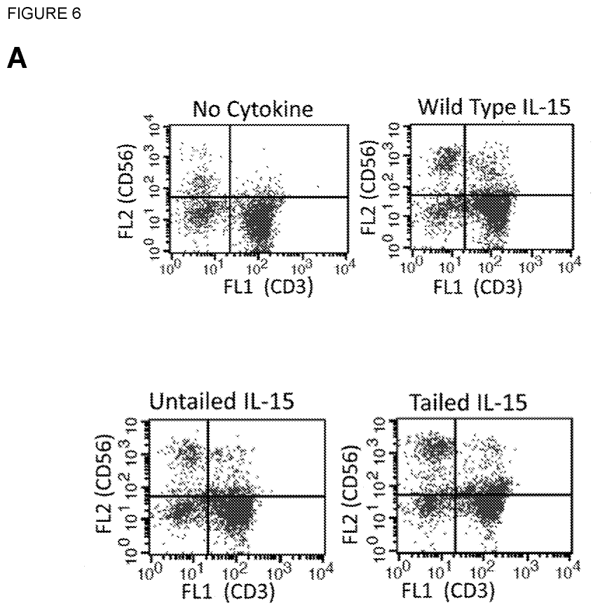

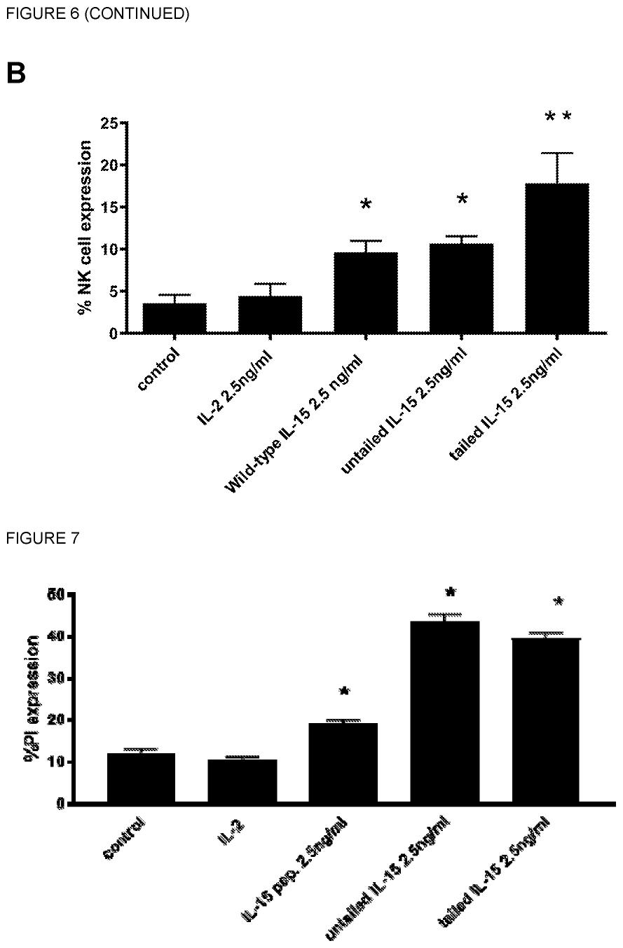

The activity of the tailed IL-15 was also confirmed using human and murine NK lymphocytes, which were incubated with tailed and untailed IL-15 to induce their expansion. After 7 days of culture, the NK cell population was analysed by flow cytometry showing that the tailed IL-15 has a greater ability to expand human NK cells (** p<0.05 compared with untailed IL-15 or wild-type IL-15 by one-way ANOVA and Newman-Keuls post-test, n=5) (FIG. 6).

### Example 6

Killing of Prostate Cancer Cells by Modified IL-15 and Membrane-Anchored Modified IL-15

Both the modified IL-15 (untailed) and membrane-anchored modified IL-15 (tailed) advantageously activated NK cell mediated killing of human prostate cancer cells when compared to the unmodified wild-type (IL-15 pep.) and IL-2 (see FIG. 7). These data confirm that both the modified IL-15 containing the IL-15 activity-promoting sequence (without a membrane-anchor) and the membrane-anchored modified IL-15 are efficacious against cancer cells, especially prostate cancer cells, thereby confirming therapeutic efficacy.

### Example 7

Study of the Activity of Modified IL-15 Polypeptides In Vivo

The efficacy of modified IL-15 polypeptides of the invention to inhibit tumour growth was further confirmed in an in vivo subcutaneous prostate cancer model in C57BL/6 mice. Male 6-8 week-old C57BL/6 mice were subcutaneously injected with 5×10⁶ TRAMP-C2 tumour cells in sterile PBS. When tumours reached 100 mm³, the mice were injected intratumourally with sterile PBS (Vehicle, n=10), modified IL-15 “untailed IL-15” (n=10), membrane-anchored modified IL-15 “tailed IL-15” (n=10), or with modified IL-15 “untailed IL-15” intraperitoneally (i.p.) (n=6). Tumour growth was measured up to 3 times per week until tumours reached a maximum diameter of 15 mm, at which stage animals were culled.

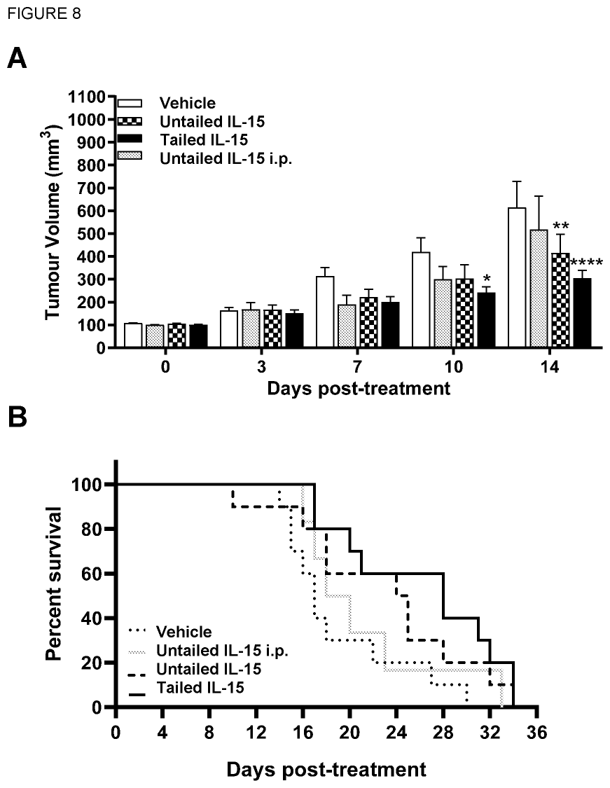

Intratumoural injection of membrane-anchored modified IL-15 “tailed IL-15” and modified IL-15 “untailed IL-15” led to a reduction (50% and 32%, respectively) of tumour growth on day 14 compared with vehicle injection. Intraperitoneal injection of modified IL-15 “untailed IL-15” reduced tumour growth by 16% compared with vehicle (FIG. 8A).

Both membrane-anchored modified IL-15 “tailed IL-15” and modified IL-15 “untailed IL-15” increased survival. Membrane-anchored modified IL-15 “tailed IL-15” significantly increased survival to 28 days compared with 17 days in the vehicle group. Modified IL-15 “untailed IL-15” increased survival to 25 days when injected intratumourally and to 19 days when injected i.p (FIG. 8B).

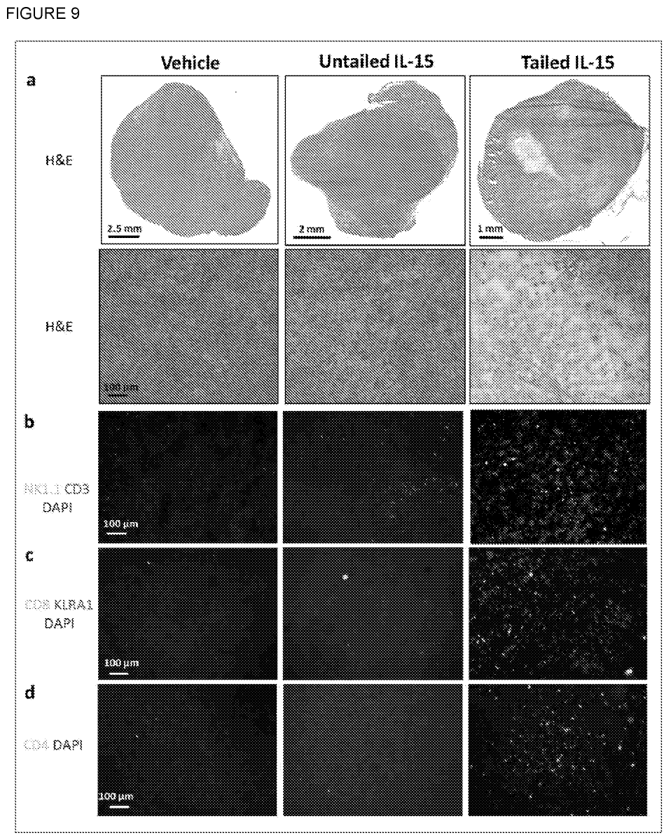

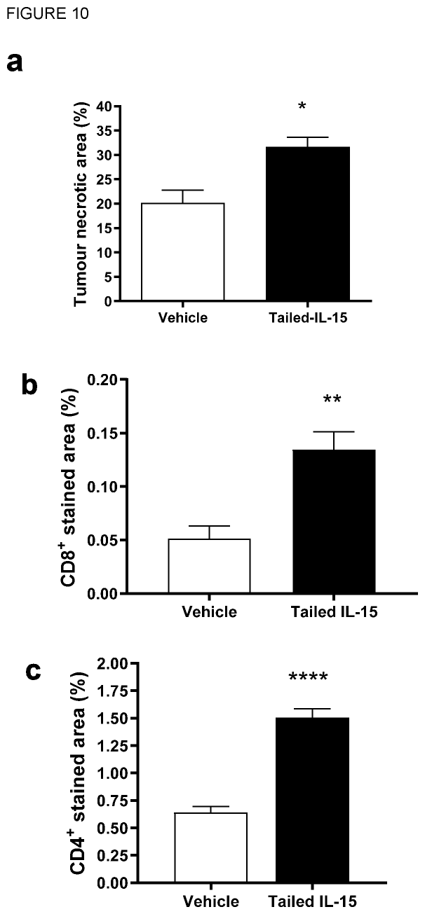

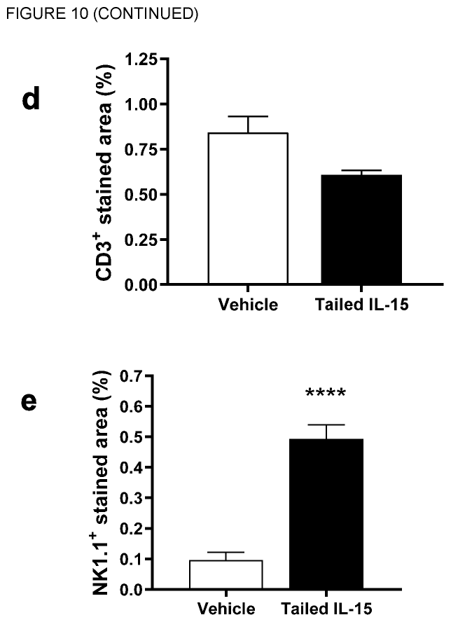

Histological analysis of the tumour tissue obtained from the animals showed increased necrosis as seen with H&E staining and increased infiltration of NK cells, CD4 and CD8 T in those animals treated with membrane-anchored modified IL-15 “tailed IL-15” and modified IL-15 “untailed IL-15” compared with PBS groups (FIG. 9). The results were particularly striking for membrane-anchored modified IL-15 “tailed IL-15”, as seen by the quantification provided in FIG. 10.

### Example 8

Alternative Modified IL-15 Polypeptides

Alternative C-terminal extensions were fused to IL-15 and their activity in the CTLL-2 assay compared to SEQ ID NO: 28 and wild-type IL-15.

The first construct was formed by fusing IL-15 to an 11 amino acid sequence (SEQ ID NO: 9) yielding fusion polypeptide SEQ ID NO: 10. The second (comparative) construct was formed by fusing IL-15 to a 67 amino acid sequence (SEQ ID NO: 11) yielding comparative fusion polypeptide SEQ ID NO: 12.

The fusion polypeptides were expressed and purified and subsequently tested in the CTLL-2 activity assay as per Example 1.

### Results

The proteins were compared using the concentrations of protein as calculated using the IL-15 Elisa Max from Biolegend (London UK) according to the manufacturer's instructions. The IL-15 Elisa measures the IL-15 in the sample that is conformationally correct (i.e. that is recognised by an IL-15 antibody).

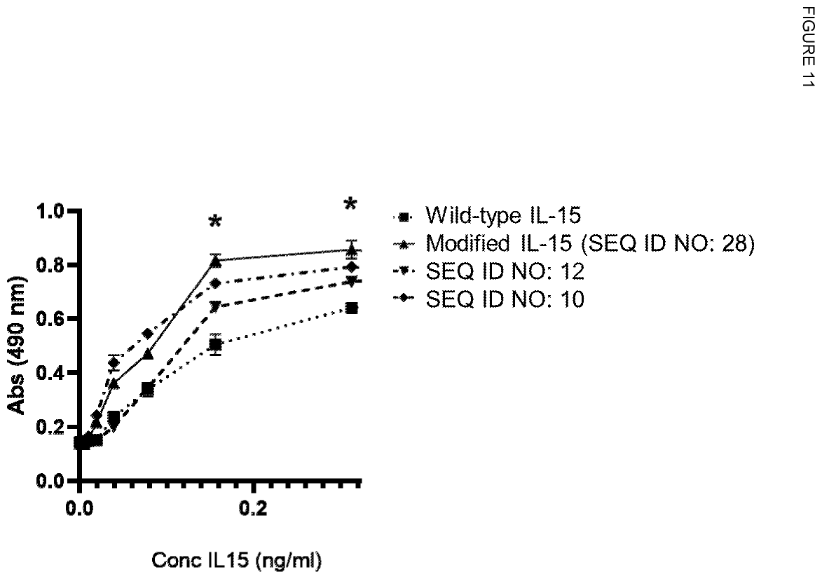

FIG. 11 shows the activity of SEQ ID NO: 28 compared with SEQ ID NO: 10 and SEQ ID NO: 12, as well as unmodified wild-type IL-15 (Peprotech, UK) in a CTLL-2 assay. SEQ ID NO: 28 is significantly more active compared to the other three proteins, while the construct containing an 11 amino acid C-terminal extension (SEQ ID NO: 10) also showed improved activity versus wild-type IL-15 and comparative construct SEQ ID NO: 12. Thus the 11 amino acid residue sequence also functioned as an IL-15 activity promoting sequence, while the fusion comprising the 67 amino acid residue sequence displayed activity similar to that of wild-type IL-15.

### Example 9

Binding of Modified IL-15 to its Receptor

To compare binding of SEQ ID NO: 28 and wild-type IL-15 to CTLL-2 cells, the proteins were labelled with Fluorescein isothiocyanate. Briefly, 100 μg of protein prepared at a concentration of 4 mg/ml was dialysed against 200 mM carbonate buffer pH 9.3 for 2 hours; FITC solution prepared at 1 mg/ml was slowly added to IL-15, until an amount of 100 ng for every 1 μg of protein was achieved and IL-15 was then incubated for 2 h with slow rotation at 4° C. A PD10 column was then used to separate free FITC from bound FITC. The protein and FITC concentrations were measured by IL-15 ELISA and Abs Max 495 nm in a spectrophotometer. A 1% solution of BSA was added to the FITC labelled proteins to stabilise the conjugation.

CTLL-2 cells were maintained by culturing with 10% TSTIM reagent (Thermofisher, UK). One hundred μl of cells at a concentration of 1×10⁶ cells per ml were aliquoted into 96 well plates with the 10% TSTIM reagent and then after 24 hrs, cells were washed twice with 0.2M glycine buffer/0.15 M NaCl (pH=3), followed by a 10 min incubation time, and then a PBS wash. Cells were then blocked for 15 mins with Fc Block (BD biosciences, UK) and then incubated for a further 30 mins with varying concentrations of FITC conjugated SEQ ID NO: 28 or FITC conjugated wild-type IL-15 at 4° C. in PBS containing 0.1% sodium azide. Cells were then washed with PBS and fixed with BD Cytofix (BD Biosciences, UK). Fluorescence intensity of the bound IL-15 was measured on a FACs Calibur flow cytometer (BD Biosciences, UK).

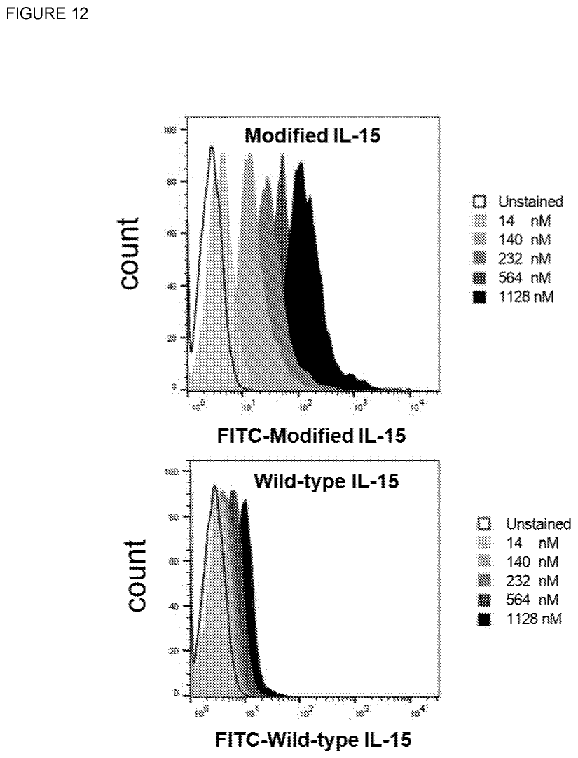

Results are presented in FIG. 12, which shows that by adding the activity-promoting sequence to IL-15, the modified IL-15 of the invention exhibits improved binding to its receptor when compared to wild-type IL-15.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A fusion polypeptide, the polypeptide comprising:
a. an interleukin-15 (IL-15); and
b. an IL-15 activity-promoting sequence, wherein said sequence:
is between 10 and 60 amino acid residues in length; and
increases CD8+ T-cell proliferation by the IL-15.

2. The fusion polypeptide according to claim 1, wherein the IL-15 activity-promoting sequence does not increase receptor-independent binding of the polypeptide to a cell surface.

3. The fusion polypeptide according to claim 1 or 2, wherein the IL-15 activity-promoting sequence is between 10 and 55 amino acid residues in length.

4. The fusion polypeptide according to any one of the preceding claims, wherein the IL-15 activity-promoting sequence is between 15 and 55 amino acid residues in length.

5. The fusion polypeptide according to any one of the preceding claims, wherein the IL-15 activity-promoting sequence is between 25 and 55 amino acid residues in length.

6. The fusion polypeptide according to any one of the preceding claims, wherein the IL-15 activity-promoting sequence is between 30 and 55 amino acid residues in length.

7. The fusion polypeptide according to any one of the preceding claims, wherein the IL-15 activity-promoting sequence is between 42 and 50 amino acid residues in length.

8. The fusion polypeptide according to any one of the preceding claims, wherein the polypeptide comprises a N-terminal IL-15 and a C-terminal IL-15 activity-promoting sequence.

9. The fusion polypeptide according to any one of the preceding claims, wherein the IL-15 activity-promoting sequence comprises a polypeptide sequence having at least 70% sequence identity to SEQ ID NO: 4 or 9.

10. The fusion polypeptide according to any one of the preceding claims, wherein the IL-15 activity-promoting sequence comprises a polypeptide sequence having at least 80% sequence identity to SEQ ID NO: 4 or 9.

11. The fusion polypeptide according to any one of the preceding claims, wherein the IL-15 activity-promoting sequence comprises a polypeptide sequence having at least 90% sequence identity to SEQ ID NO: 4 or 9.

12. The fusion polypeptide according to any one of the preceding claims, wherein the IL-15 activity-promoting sequence comprises a polypeptide sequence having at least 95% sequence identity to SEQ ID NO: 4 or 9.

13. The fusion polypeptide according to any one of the preceding claims, wherein the IL-15 activity-promoting sequence comprises SEQ ID NO: 4 or 9.

14. The fusion polypeptide according to any one of the preceding claims, wherein the IL-15 activity-promoting sequence consists of SEQ ID NO: 4 or 9.

15. The fusion polypeptide according to any one of the preceding claims, wherein the IL-15 is a human IL-15.

16. The fusion polypeptide according to any one of the preceding claims, wherein the IL-15 comprises a polypeptide sequence having at least 70% sequence identity to SEQ ID NO: 2 or 3.

17. The fusion polypeptide according to any one of the preceding claims comprising a polypeptide sequence having at least 70% sequence identity to SEQ ID NO: 5, 10 or 28.

18. The fusion polypeptide according to any one of the preceding claims comprising a polypeptide sequence having at least 80% sequence identity to SEQ ID NO: 5, 10 or 28.

19. The fusion polypeptide according to any one of the preceding claims comprising a polypeptide sequence having at least 90% sequence identity to SEQ ID NO: 5, 10 or 28.

20. The fusion polypeptide according to any one of the preceding claims comprising a polypeptide sequence having at least 95% sequence identity to SEQ ID NO: 5, 10 or 28.

21. The fusion polypeptide according to any one of the preceding claims comprising SEQ ID NO: 5, 10 or 28.

22. The polypeptide according to any one of the preceding claims, wherein a membrane binding agent is conjugated to the IL-15 activity-promoting sequence.

23. The fusion polypeptide according to claim 22, wherein the membrane binding agent comprises an aliphatic acyl group.

24. The fusion polypeptide according to claim 23, wherein the aliphatic acyl group is myristoyl.

25. The fusion polypeptide according to any one of claims 22-24, wherein the membrane binding element further comprises a hydrophilic peptide.

26. The fusion polypeptide according to claim 25, wherein the hydrophilic peptide comprises a peptide sequence having at least 70% sequence identity to SEQ ID NO: 6.

27. The fusion polypeptide according to claim 25 or 26, wherein the hydrophilic peptide comprises a peptide sequence having at least 80% sequence identity to SEQ ID NO: 6.

28. The fusion polypeptide according to any one of claims 25-27, wherein the hydrophilic peptide comprises a peptide sequence having at least 90% sequence identity to SEQ ID NO: 6.

29. The fusion polypeptide according to any one of claims 25-28, wherein the hydrophilic peptide comprises a peptide sequence having at least 95% sequence identity to SEQ ID NO: 6.

30. The fusion polypeptide according to any one of claims 25-29, wherein the hydrophilic peptide comprises SEQ ID NO: 6.

31. The fusion polypeptide according to any one of claims 25-30, wherein the hydrophilic peptide consists of SEQ ID NO: 6.

32. The fusion polypeptide according to any one of claims 22-31, wherein the membrane binding element is conjugated to a cysteine residue or a lysine residue of the IL-15 activity-promoting sequence.

33. The fusion polypeptide according to any one of the preceding claims, wherein the fusion polypeptide is conjugated to N-(α,εbis-myristoyllysine)SSKSPSKKDDKKPGDC (SEQ ID NO: 31) via a di-sulphide bond.

34. A fusion polypeptide, the polypeptide comprising:
a. an interleukin-15 (IL-15); and
b. a peptide, wherein the peptide is between 10 and 60 amino acid residues in length and has at least 70% sequence identity to SEQ ID NO: 4 or 9.

35. The fusion polypeptide according to claim 34, wherein the peptide is between 10 and 55 amino acid residues in length.

36. The fusion polypeptide according to claim 34 or 35, wherein the peptide is between 15 and 55 amino acid residues in length.

37. The fusion polypeptide according to any one of claims 34-36, wherein the peptide is between 25 and 55 amino acid residues in length.

38. The fusion polypeptide according to any one of claims 34-37, wherein the peptide is between 30 and 55 amino acid residues in length.

39. The fusion polypeptide according to any one of claims 34-38, wherein the peptide is between 42 and 50 amino acid residues in length.

40. The fusion polypeptide according to any one of claims 34-39, wherein the polypeptide comprises a N-terminal IL-15 and a C-terminal peptide.

41. The fusion polypeptide according to any one of claims 34-40, wherein the peptide comprises a polypeptide sequence having at least 80% sequence identity to SEQ ID NO: 4 or 9.

42. The fusion polypeptide according to any one of claims 34-41, wherein the peptide comprises a polypeptide sequence having at least 90% sequence identity to SEQ ID NO: 4 or 9.

43. The fusion polypeptide according to any one of claims 34-42, wherein the peptide comprises a polypeptide sequence having at least 95% sequence identity to SEQ ID NO: 4 or 9.

44. The fusion polypeptide according to any one of claims 34-43, wherein the peptide comprises SEQ ID NO: 4 or 9 (preferably consists of SEQ ID NO: 4 or 9).

45. The fusion polypeptide according to any one of claims 34-44, wherein the IL-15 is a human IL-15.

46. The fusion polypeptide according to any one of claims 34-45, wherein the IL-15 comprises a polypeptide sequence having at least 70% sequence identity to SEQ ID NO: 2 or 3.

47. The fusion polypeptide according to any one of claims 34-46 comprising a polypeptide sequence having at least 70% sequence identity to SEQ ID NO: 5, 10 or 28.

48. The fusion polypeptide according to any one of claims 34-47 comprising a polypeptide sequence having at least 80% sequence identity to SEQ ID NO: 5, 10 or 28.

49. The fusion polypeptide according to any one of claims 34-48 comprising a polypeptide sequence having at least 90% sequence identity to SEQ ID NO: 5, 10 or 28.

50. The fusion polypeptide according to any one of claims 34-49 comprising a polypeptide sequence having at least 95% sequence identity to SEQ ID NO: 5, 10 or 28.

51. The fusion polypeptide according to any one of claims 34-50 comprising SEQ ID NO: 5, 10 or 28.

52. The polypeptide according to any one of claims 34-51, wherein a membrane binding agent is conjugated to the peptide.

53. The fusion polypeptide according to claim 52, wherein the membrane binding agent comprises an aliphatic acyl group.

54. The fusion polypeptide according to claim 53, wherein the aliphatic acyl group is myristoyl.

55. The fusion polypeptide according to any one of claims 52-54, wherein the membrane binding element further comprises a hydrophilic peptide.

56. The fusion polypeptide according to claim 55, wherein the hydrophilic peptide comprises a peptide sequence having at least 70% sequence identity to SEQ ID NO: 6.

57. The fusion polypeptide according to claim 55 or 56, wherein the hydrophilic peptide comprises a peptide sequence having at least 80% sequence identity to SEQ ID NO: 6.

58. The fusion polypeptide according to any one of claims 55-57, wherein the hydrophilic peptide comprises a peptide sequence having at least 90% sequence identity to SEQ ID NO: 6.

59. The fusion polypeptide according to any one of claims 55-58, wherein the hydrophilic peptide comprises a peptide sequence having at least 95% sequence identity to SEQ ID NO: 6.

60. The fusion polypeptide according to any one of claims 55-59, wherein the hydrophilic peptide comprises SEQ ID NO: 6.

61. The fusion polypeptide according to any one of claims 55-60, wherein the hydrophilic peptide consists of SEQ ID NO: 6.

62. The fusion polypeptide according to any one of claims 52-61, wherein the membrane binding element is conjugated to a cysteine residue or a lysine residue of the peptide.

63. The fusion polypeptide according to any one of claims 34-62, wherein the fusion polypeptide is conjugated to N-(α,εbis-myristoyllysine)SSKSPSKKDDKKPGDC (SEQ ID NO: 31) via a di-sulphide bond.

64. A fusion polypeptide, the polypeptide comprising a polypeptide sequence having at least 70% sequence identity to SEQ ID NO: 5, 10 or 28.

65. The fusion polypeptide according to claim 64, wherein the IL-15 is a human IL-15.

66. The fusion polypeptide according to claim 64 or 65, wherein the IL-15 comprises a polypeptide sequence having at least 80% sequence identity to SEQ ID NO: 2 or 3.

67. The fusion polypeptide according to any one of claims 64-66 comprising a polypeptide sequence having at least 90% sequence identity to SEQ ID NO: 5, 10 or 28.

68. The fusion polypeptide according to any one of claims 64-67 comprising a polypeptide sequence having at least 95% sequence identity to SEQ ID NO: 5, 10 or 28.

69. The fusion polypeptide according to any one of claims 64-68 comprising a polypeptide sequence comprising SEQ ID NO: 5, 10 or 28.

70. The fusion polypeptide according to any one of claims 64-69 consisting of SEQ ID NO: 5, 10 or 28.

71. The polypeptide according to any one of claims 64-70, wherein a membrane binding agent is conjugated to the polypeptide.

72. The fusion polypeptide according to claim 71, wherein the membrane binding agent comprises an aliphatic acyl group.

73. The fusion polypeptide according to claim 72, wherein the aliphatic acyl group is myristoyl.

74. The fusion polypeptide according to any one of claims 71-73, wherein the membrane binding element further comprises a hydrophilic peptide.

75. The fusion polypeptide according to claim 74, wherein the hydrophilic peptide comprises a peptide sequence having at least 70% sequence identity to SEQ ID NO: 6.

76. The fusion polypeptide according to claim 74 or 75, wherein the hydrophilic peptide comprises a peptide sequence having at least 80% sequence identity to SEQ ID NO: 6.

77. The fusion polypeptide according to any one of claims 74-76, wherein the hydrophilic peptide comprises a peptide sequence having at least 90% sequence identity to SEQ ID NO: 6.

78. The fusion polypeptide according to any one of claims 74-77, wherein the hydrophilic peptide comprises a peptide sequence having at least 95% sequence identity to SEQ ID NO: 6.

79. The fusion polypeptide according to any one of claims 74-78, wherein the hydrophilic peptide comprises SEQ ID NO: 6.

80. The fusion polypeptide according to any one of claims 74-79, wherein the hydrophilic peptide consists of SEQ ID NO: 6.

81. The fusion polypeptide according to any one of claims 71-80, wherein the membrane binding element is conjugated to a cysteine residue or a lysine residue of the polypeptide.

82. The fusion polypeptide according to any one of claims 64-81, wherein the fusion polypeptide is conjugated to N-(α,εbis-myristoyllysine)SSKSPSKKDDKKPGDC (SEQ ID NO: 31) via a di-sulphide bond.

83. The fusion polypeptide according to any one of the preceding claims, wherein the fusion polypeptide is encoded by a nucleotide sequence having at least 70% sequence identity to SEQ ID NO: 8 or SEQ ID NO: 24.

84. The fusion polypeptide according to any one of the preceding claims, wherein the fusion polypeptide is encoded by a nucleotide sequence having at least 80% sequence identity to SEQ ID NO: 8 or SEQ ID NO: 24.

85. The fusion polypeptide according to any one of the preceding claims, wherein the fusion polypeptide is encoded by a nucleotide sequence having at least 90% sequence identity to SEQ ID NO: 8 or SEQ ID NO: 24.

86. The fusion polypeptide according to any one of the preceding claims, wherein the fusion polypeptide is encoded by a nucleotide sequence having at least 95% sequence identity to SEQ ID NO: 8 or SEQ ID NO: 24.

87. The fusion polypeptide according to any one of the preceding claims, wherein the fusion polypeptide is encoded by a nucleotide sequence comprising SEQ ID NO: 8 or SEQ ID NO: 24.

88. The fusion polypeptide according to any one of the preceding claims, wherein the fusion polypeptide is encoded by a nucleotide sequence consisting of SEQ ID NO: 8 or SEQ ID NO: 24.

89. The fusion polypeptide according to any one of the preceding claims, wherein the fusion polypeptide comprises a polypeptide sequence having at last 70% sequence identity to SEQ ID NO: 7.

90. The fusion polypeptide according to any one of the preceding claims, wherein the fusion polypeptide comprises a polypeptide sequence having at last 80% sequence identity to SEQ ID NO: 7.

91. The fusion polypeptide according to any one of the preceding claims, wherein the fusion polypeptide comprises a polypeptide sequence having at last 90% sequence identity to SEQ ID NO: 7.

92. The fusion polypeptide according to any one of the preceding claims, wherein the fusion polypeptide comprises a polypeptide sequence having at last 95% sequence identity to SEQ ID NO: 7.

93. The fusion polypeptide according to any one of the preceding claims, wherein the fusion polypeptide comprises SEQ ID NO: 7.

94. The fusion polypeptide according to any one of the preceding claims, wherein the fusion polypeptide consists of SEQ ID NO: 7.

95. A nucleic acid encoding a fusion polypeptide according to any one of claims 1-94.

96. The nucleic acid according to claim 95, wherein the nucleic acid comprises a nucleotide sequence having at least 70% sequence identity to SEQ ID NO: 8 or SEQ ID NO: 24.

97. The nucleic acid according to claim 95 or 96, wherein the nucleic acid comprises a nucleotide sequence having at least 80% sequence identity to SEQ ID NO: 8 or SEQ ID NO: 24.

98. The nucleic acid according to any one of claims 95-97, wherein the nucleic acid comprises a nucleotide sequence having at least 90% sequence identity to SEQ ID NO: 8 or SEQ ID NO: 24.

99. The nucleic acid according to any one of claims 95-98, wherein the nucleic acid comprises a nucleotide sequence having at least 95% sequence identity to SEQ ID NO: 8 or SEQ ID NO: 24.

100. The nucleic acid according to any one of claims 95-99, wherein the nucleic acid comprises SEQ ID NO: 8 or SEQ ID NO: 24.

101. The nucleic acid according to any one of claims 95-100, wherein the nucleic acid comprises a nucleotide sequence consists of SEQ ID NO: 8 or SEQ ID NO: 24.

102. A method for producing a fusion polypeptide, the method comprising:
a. expressing the nucleic acid sequence according to any one of claims 95-101 in a host cell; and
b. isolating the fusion polypeptide.

103. A fusion polypeptide obtainable by the method of claim 102.

104. A pharmaceutical composition comprising the fusion polypeptide according to any one of claim 1-94 or 103 and a pharmaceutically acceptable carrier, excipient, adjuvant, and/or salt.

105. A kit comprising:
a. the fusion polypeptide according to any one of claim 1-94 or 103 or the pharmaceutical composition according to claim 104; and
b. instructions for use of the same (e.g. in treating cancer).

106. A fusion polypeptide according to any one of claim 1-94 or 103 or a pharmaceutical composition according to claim 104 or a kit according to claim 105 for use in treating cancer.

107. A method of treating cancer, the method comprising administering a fusion polypeptide according to any one of claim 1-94 or 103 or a pharmaceutical composition according to claim 104 or a kit according to claim 105 to a subject.

108. Use of a fusion polypeptide according to any one of claim 1-94 or 103 or a pharmaceutical composition according to claim 104 or a kit according to claim 105 in the manufacture of a medicament for treating cancer.

109. The fusion polypeptide, pharmaceutical composition, or kit for use, method or use according to any one of claims 106-108, wherein the cancer is a solid tumour cancer.

110. The fusion polypeptide, pharmaceutical composition, or kit for use, method or use according to any one of claims 106-109, wherein the cancer is one or more selected from: prostate cancer, colon cancer, breast cancer, lung cancer, skin cancer, liver cancer, bone cancer, ovarian cancer, pancreatic cancer, brain cancer, head cancer, neck cancer, lymphoma, and neuronal cancer.

111. The fusion polypeptide, pharmaceutical composition, or kit for use, method, or use according to any one of claims 106-110, wherein the fusion polypeptide or composition is administered intratumourally.


SEQUENCE LISTING (32 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 115 aa, Homo sapiens
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 46 aa, Artificial Sequence
  Activity-Promoting Sequence
GSGSRGKSLTSKVPPTVQKPTTVNVPTTEVSPTSQKTTTHHHHHHC

SEQ ID NO: 5 - protein, 161 aa, Artificial Sequence
  Fusion Polypeptide
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSRGKSLTSKVPPTVQKPTTVNVPTTEVSPTSQKTTTHHHHHHC

SEQ ID NO: 6 - protein, 16 aa, Artificial Sequence
  Hydrophilic Peptide
SSKSPSKKDDKKPGDC

SEQ ID NO: 7 - protein, 161 aa, Artificial Sequence; identical to SEQ ID NO: 5
  Fusion Polypeptide Comprising a Membrane Binding Element
  MISC_FEATURE (161)..(161): Disulfide bonded to cysteine of membrane binding element (SEQ ID NO 31)

SEQ ID NO: 8 - DNA, 477 bp, Artificial Sequence
  Nucleic Acid Sequence Encoding SEQ ID NO 28
aactgggtgaacgttatctcggacctgaaaaaaatcgaagacctgatccaaagcatgcacattgacgctacgctgtatacggaaagcgatgtgcatccgtcgtgcaaagttaccgcgatgaaatgttttctgctggaactgcaggtcatttcgctggaaagcggcgatgcgagtatccacgacaccgttgaaaacctgattatcctggccaacaattccctgagctctggcaatgtgacggaatcaggttgcaaagaatgtgaagaactggaagagaaaaacatcaaagaattcctgcagtctttcgtccatattgtgcaaatgttcatcaatacgagtggctccggttcacgtggtaaatctctgaccagtaaagttccgccgacggtccaaaaaccgaccacggtgaacgttccgaccaccgaagtctctccgaccagtcagaaaaccaccacccaccatcaccatcatcattgc

SEQ ID NO: 9 - protein, 11 aa, Artificial Sequence
  Activity-Promoting Sequence 2
GSGSHHHHHHC

SEQ ID NO: 10 - protein, 125 aa, Artificial Sequence
  Fusion Polypeptide 2
MNWVNVISDLKKIEDLIQSMHIDATLYTESDVHPSCKVTAMKCFLLELQVISLESGDASIHDTVENLIILANNSLSSGNVTESGCKECEELEEKNIKEFLQSFVHIVQMFINTSGSGSHHHHHHC

SEQ ID NO: 11 - protein, 67 aa, Artificial Sequence
  Comparative Fusion Sequence
GSGSRGKSLTSKVPPTVQKPTTVNVPTTEVSPTSQKTTTKTTTPNAQATRSTPVSRTTKHHHHHHHC

SEQ ID NO: 12 - protein, 181 aa, Artificial Sequence
  Comparative Fusion Polypeptide
MNWVNVISDLKKIEDLIQSMHIDATLYTESDVHPSCKVTAMKCFLLELQVISLESGDASIHDTVENLIILANNSLSSGNVTESGCKECEELEEKNIKEFLQSFVHIVQMFINTSGSGSRGKSLTSKVPPTVQKPTTVNVPTTEVSPTSQKTTTKTTTPNAQATRSTPVSRTTKHHHHHHHC

SEQ ID NO: 13 - protein, 125 aa, Artificial Sequence; identical to SEQ ID NO: 10
  Fusion Polypeptide 2 Comprising a Membrane Binding Element
  MISC_FEATURE (125)..(125): Disulfide bonded to cysteine of membrane binding element (SEQ ID NO 31)

SEQ ID NO: 14 - protein, 6 aa, Homo sapiens
GRGDSP

SEQ ID NO: 15 - protein, 13 aa, Homo sapiens
DGPSEILRGDFSS

SEQ ID NO: 16 - protein, 17 aa, Homo sapiens
GNEQSFRVDLRTLLRYA

SEQ ID NO: 17 - protein, 15 aa, Homo sapiens
SAAPSSGFRILLLKV

SEQ ID NO: 18 - protein, 17 aa, Homo sapiens
AAPSVIGFRILLLKVAG

SEQ ID NO: 19 - protein, 16 aa, Artificial Sequence
  Hydrophilic Peptide
DGPKKKKKKSPSKSSG

SEQ ID NO: 20 - protein, 16 aa, Artificial Sequence
  Hydrophilic Peptide
GSSKSPSKKKKKKPGD

SEQ ID NO: 21 - protein, 20 aa, Artificial Sequence
  Hydrophilic Peptide
SPSNETPKKKKKRFSFKKSG

SEQ ID NO: 22 - protein, 16 aa, Artificial Sequence
  Hydrophilic Peptide
DGPKKKKKKSPSKSSK

SEQ ID NO: 23 - protein, 14 aa, Artificial Sequence
  Hydrophilic Peptide
SKDGKKKKKKSKTK

SEQ ID NO: 24 - DNA, 480 bp, Artificial Sequence
  Nucleic Acid Sequence Encoding SEQ ID NO 28 plus Met
atgaactgggtgaacgttatctcggacctgaaaaaaatcgaagacctgatccaaagcatgcacattgacgctacgctgtatacggaaagcgatgtgcatccgtcgtgcaaagttaccgcgatgaaatgttttctgctggaactgcaggtcatttcgctggaaagcggcgatgcgagtatccacgacaccgttgaaaacctgattatcctggccaacaattccctgagctctggcaatgtgacggaatcaggttgcaaagaatgtgaagaactggaagagaaaaacatcaaagaattcctgcagtctttcgtccatattgtgcaaatgttcatcaatacgagtggctccggttcacgtggtaaatctctgaccagtaaagttccgccgacggtccaaaaaccgaccacggtgaacgttccgaccaccgaagtctctccgaccagtcagaaaaccaccacccaccatcaccatcatcattgc

SEQ ID NO: 25 - protein, 161 aa, Artificial Sequence
  Full-Length Interleukin-15 Variant
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSGNVTESGCKECEELEEKNIKEFLQSFVHIVQMFINTS

SEQ ID NO: 26 - protein, 113 aa, Artificial Sequence
  Mature Interleukin-15 - Amino Acids 49-162 Variant
NWVNVISDLKKIEDLIQSMHIDATLYTESDVHPSCKVTAMKCFLLELQVISLESGDASIHDTVENLIILANNSLSSGNVTESGCKECEELEEKNIKEFLQSFVHIVQMFINTS

SEQ ID NO: 27 - protein, 114 aa, Artificial Sequence
  Mature Interleukin-15 Variant
MNWVNVISDLKKIEDLIQSMHIDATLYTESDVHPSCKVTAMKCFLLELQVISLESGDASIHDTVENLIILANNSLSSGNVTESGCKECEELEEKNIKEFLQSFVHIVQMFINTS

SEQ ID NO: 28 - protein, 160 aa, Artificial Sequence
  Fusion Polypeptide
MNWVNVISDLKKIEDLIQSMHIDATLYTESDVHPSCKVTAMKCFLLELQVISLESGDASIHDTVENLIILANNSLSSGNVTESGCKECEELEEKNIKEFLQSFVHIVQMFINTSGSGSRGKSLTSKVPPTVQKPTTVNVPTTEVSPTSQKTTTHHHHHHC

SEQ ID NO: 29 - protein, 160 aa, Artificial Sequence; identical to SEQ ID NO: 28
  Fusion Polypeptide Comprising a Membrane Binding Element
  MISC_FEATURE (160)..(160): Disulfide bonded to cysteine of membrane binding element (SEQ ID NO 31)

SEQ ID NO: 30 - protein, 17 aa, Artificial Sequence
  Membrane Binding Element
  MISC_FEATURE (1)..(1): Xaa is alpha, epsilon bis-myristoyl lysine
  MISC_FEATURE (17)..(17): Xaa is (S-2-pyridyldithio)-C-acid
XSSKSPSKKDDKKPGDX

SEQ ID NO: 31 - protein, 17 aa, Artificial Sequence
  Membrane Binding Element
  MISC_FEATURE (1)..(1): Xaa is alpha, epsilon bis-myristoyl lysine
XSSKSPSKKDDKKPGDC

SEQ ID NO: 32 - protein, 9 aa, Homo sapiens
GFRILLLKV